SEQUENCE LISTING (2 sequences)

SEQ ID NO: 1 - DNA, 22 bp, synthetic construct
  source 1..22: mol_type = other DNA
tcgtcgttgtcgttttgtcgtt

SEQ ID NO: 2 - DNA, 14 bp, synthetic construct
  source 1..14: mol_type = other DNA
tcgtcgttgtcgtt

(19) United States
(12) Patent Application Publication
(10) Pub. No.: US 2026/0232784 A1
(43) Pub. Date: Aug. 13, 2026

(54) METHODS OF ELICITING AN IMMUNE RESPONSE AGAINST CLOSTRIDIUM PERFRINGENS AND/OR NECROTIC ENTERITIS AND COMPOSITIONS FOR USE THEREOF

(71) Applicant: University of Saskatchewan, Saskatoon (CA)

(72) Inventors: Suresh TIKOO, Saskatoon (CA); Hemlata GAUTAM, Saskatoon (CA); Susantha Muhandiramge GOMIS, Saskatoon (CA)

(21) Appl. No.: 19/151,264

(22) PCT Filed: Jan. 26, 2024
(86) PCT No.: PCT/CA2024/050096

§ 371 Date: Jul. 25, 2025

Related U.S. Application Data
(60) Provisional application No. 63/441,658, filed on Jan. 27, 2023.

(51) Int. Cl. A61K 39/08; A61K 39/00; A61K 39/39; A61P 37/04; C12N 15/117

(52) U.S. Cl. CPC A61K 39/08; A61K 39/39; A61P 37/04; C12N 15/117; A61K 2039/521; A61K 2039/544; A61K 2039/552

(57) ABSTRACT

Provided herein are methods of eliciting an immune response in one or more subject(s) against Clostridium perfringens (C. perfringens) infection and/or necrotic enteritis, the method comprising administering to one or more subject(s) an amount of a composition comprising live C. perfringens and compositions for such use.

### CROSS-REFERENCE TO RELATED APPLICATIONS

This application claims the benefit of priority to U.S. Provisional Application No. 63/441,658 filed Jan. 27, 2023, the contents of which are incorporated herein by reference in its entirety.

### INCORPORATION OF SEQUENCE LISTING

A computer readable form of the Sequence Listing “P69278PC00_SListing.xml” (2,637 bytes) created on Jan. 26, 2024, is herein incorporated by reference.

### FIELD

The present disclosure relates to compositions and methods of eliciting an immune response against Clostridium perfringens and/or necrotic enteritis and in particular compositions comprising live C. perfringens.

### BACKGROUND

Necrotic enteritis (NE) is one of the most important emerging diseases in the broiler chicken industry worldwide. NE led to severe production and economic losses to poultry industry and estimated to be at approximately $6 billion USD annually (11). Clostridium perfringens (CP) proliferates in the intestine and produces exotoxins that causes necrosis of the gut resulting in death. CP is transmitted by faeco-oral route and part of normal flora of the intestines as well (<1%). The incidence of NE has significantly increased because of decreased usage of prophylactic antibiotics in poultry feed amid concerns related to the emergence of antibiotic-resistant superbugs (6). NE usually occurs in 2-6-week-old broiler chickens (1). Birds exhibiting clinical signs usually die within a few hours (2). Mortality rates associated with NE may range from 2 to 50% in broilers ((7),(6)). Necrotic toxin B-like (netB) and α-toxin have been identified as virulence factors (3). Although evidence exists associating netB with NE development (3), not all CP isolates from NE outbreaks were netB-positive (9). There is an urgent need to investigate alternative approaches for controlling NE (8).

Predisposing factors for NE: NE is a complex disease and preventing this disease is a challenge amid antimicrobial use (AMU) reduction strategies of the poultry industry. NE is influenced by several predisposing factors, including diet, immune status, and stress. Immunosuppression caused by pathogens like variant infectious bursal disease virus (vIBDV) and coccidiosis predisposes chickens to NE (6). In a disease model of NE, it was reported that birds infected with IBDV are also frequently infected with CP (7). Previously we have reported that about 43% of farms or (52% of barns) in Saskatchewan were infected with vBDVs (12). We have also seen over 50% of farms infected with vlBDV in the Fraser Valley, BC (unpublished data). We have recently demonstrated that circulating vIBDV may pose a challenge for antibiotic-free chicken farming in Canada (4). Given a wide prevalence of vIBDV in the Canadian broiler chicken industry, a vaccination strategy that aims to control NE amid AMU reduction strategies should be evaluated for its effectiveness in the context of vlBDV-induced immunosuppression.

Current status of NE vaccines and challenges: NE vaccine studies have tested proteins and toxins as vaccine candidates, but results were inconsistent (9). A vaccine study using the toxoid of three different CP strains showed that immunity to NE is not entirely determined by netB or α-toxin, but probably involves other antigens as well (5). In the poultry industry, vaccination of breeder hens is often preferred to induce immunity in broiler chicks via maternal antibodies (MtAb). However, MtAb decline by 3 weeks of age (8). This may also explain why outbreaks of NE are mostly observed between 3 to 4 weeks of age (10). Induction of vaccine immunity in young broilers is hampered by immaturity of the immune system or inadequate sensitization of the immune system to antigens. A challenge is how to protect broilers and other poultry against NE, for example when they are susceptible between 3-6 weeks of age.

### SUMMARY

The inventors have found as disclosed herein a strategy for protecting broilers and other poultry against NE. Poultry can be susceptible to NE between 3-6 weeks of age. The inventors have found that for example, a single vaccine administration at hatch can mount sufficient immunity within the first 3 weeks of life without a booster vaccine that can provide protection for the duration of the broiler's life. Accordingly, an aspect of the disclosure includes a method of eliciting an immune response in one or more subject(s) against Clostridium perfringens (C. perfringens) infection and/or necrotic enteritis, the method comprising administering to one or more subject(s) an amount of a composition comprising live C. perfringens, and optionally a pharmaceutically acceptable carrier or diluent.

Another aspect of the disclosure includes a method of inducing protective immunity against or preventing Clostridium perfringens (C. perfringens) infection and/or necrotic enteritis, the method comprising administering to one or more subject(s) an amount of a composition comprising live C. perfringens, and optionally a pharmaceutically acceptable carrier or diluent.

In an embodiment, the amount is an immunologically effective amount.

Another aspect of the disclosure includes a composition comprising live Clostridium perfringens (C. perfringens), and optionally a pharmaceutically acceptable carrier or diluent, for use in eliciting an immune response in one or more subject(s) against C. perfringens infection and/or necrotic enteritis and/or inducing protective immunity against or preventing C. perfringens infection and/or necrotic enteritis.

Another aspect of the disclosure includes use of a composition comprising live Clostridium perfringens (C. perfringens), and optionally a pharmaceutically acceptable carrier or diluent for eliciting an immune response in one or more subject(s) against C. perfringens infection and/or necrotic enteritis and/or for inducing protective immunity against or preventing C. perfringens infection and/or necrotic enteritis.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### BRIEF DESCRIPTION OF THE DRAWINGS

Further objects, features and advantages of the disclosure will become apparent from the following detailed description taken in conjunction with the accompanying figures showing illustrative embodiments of the disclosure, in which:

FIG. 1A is a series of images depicting histopathological lesions associated with necrotic enteritis in the animal models used in the Examples described herein and their associated scoring.

FIG. 1B is series of images of gross lesions associate with necrotic enteritis in the animal models used in the Examples described herein.

FIG. 2 is a graph showing gross and histopathological lesions of NE in birds vaccinated with live or inactivated C. perfringens vaccine described herein by the intrapulmonary route.

FIG. 3 is a graph depicting mortality of different experimental groups shown as percent survival in different groups on 0, 1, 2, and 3 days post challenge with C. perfringens.

FIG. 4 is a graph showing gross and histopathological lesions of NE in birds vaccinated with live C. perfringens vaccine described herein by the intrapulmonary route. CP=C. perfringens; cha=C. perfringens challenge; ino=in ovo; Liv=live.

FIG. 5 is a series of graphs displaying flow cytometry results from analysis of IgM^{high} and IgM^{low} B cells in peripheral blood mononuclear cell (PBMC's) and spleens groups receiving only saline, the live C. perfringens vaccine described herein, or in ovo CpG-ODNs and the live C. perfringens vaccine descried herein.

FIG. 6 is a graph depicting mortality of different experimental groups shown as percent survival in different groups on 0, 1, 2, and 3 days post challenge with C. perfringens.

FIG. 7 is a graph depicting gross and histopathological lesions of NE in birds vaccinated with live CP vaccine by the intrapulmonary route compared to C. perfringens subcutaneous vaccine. [CP=C. perfringens; cha=C. perfringens challenge; ino=in ovo; Liv=live]. No histopathological NE lesions were observed in the group 1, while all birds (100%) had NE lesions in the groups 2 (CP challenge only). A significant reduction in NE lesions has been observed in vaccinated groups (p<0.0001).

FIG. 8A is an image depicting lyophilized compositions comprising E. coli and 1% sucrose (left) and 1% mannitol (right).

FIG. 8B is an image depicting lyophilized compositions comprising E. coli and 10% sucrose (left) and 10% mannitol (right).

FIG. 8C is an image depicting lyophilized compositions comprising E. coli and 25% sucrose (left) and 25% mannitol (right).

FIG. 8D is an image depicting lyophilized compositions comprising C. perfringens and 1% sucrose (left) and 1% mannitol (right).

FIG. 8E is an image depicting lyophilized compositions comprising C. perfringens and 10% sucrose (left) and 10% mannitol (right).

FIG. 8F is an image depicting lyophilized compositions comprising C. perfringens and 25% sucrose (left) and 25% mannitol (right).

FIG. 9A is an image depicting solutions of 1% sucrose (left) and 1% mannitol (right).

FIG. 9B is an image depicting solutions of 10% sucrose (left) and 10% mannitol (right).

FIG. 9C is an image depicting solutions of 25% sucrose (left) and 25% mannitol (right).

FIG. 10 is an image showing bacterial cultures showing there was no difference in the viability of C. perfringens (7×10⁹ cfu/mL) following lyophilization media either with skim milk (top) or sucrose (bottom)].

FIG. 11A is an image of live C. perfringens lyophilized with skim milk, showing the 1 mL leftover following nebulization for 15 minutes (left) compared to the right container with sucrose.

FIG. 11B is an image showing pellets of C. perfringens after lyophilization with sucrose (left) and skim milk (right).

FIG. 12A-12C depicts images of endospore staining. FIG. 12A shows live C. perfringens culture before heat inactivation. FIG. 12B shows inactivated C. perfringens following heat treatment at 65° C. for 1 hour. No spores detected and variable size of bacteria indicates the division (log phase). FIG. 12C shows C. perfringens after formaldehyde treatment.

FIG. 13A-13L are graphs depicting a comparison of metabolic outputs for OCR and ECAR of PBMCs in broiler chickens injected with both CpG-DON (in-ovo route)+vaccine (C. perfringens 1×10⁸ cfu/mL) via IPL route, vaccine only and CpG-DON (in-ovo route) only groups.

FIG. 14A-14B are graphs depicting survival and histopathological score against NE in broiler chickens following live CP vaccination by the IPL route at hatch and with or without booster CP vaccine and CpG-ODN against heterologous CP. FIG. 14A shows percent survival of broiler chickens in different groups. FIG. 14B shows histopathological score of broiler chickens in different groups. *** signifies p<0.001, and **** signifies p<0.0001.

### DETAILED DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

The following non-limiting examples are illustrative of the present application:

### 1. Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

The term “about” as used herein may be used to take into account experimental error and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “live C. perfringens” or variations thereof as used herein, refers to C. perfringens (CP) bacteria that are viable and includes wild type or naturally occurring CP as well as mutated CP, including for example attenuated CP. In some embodiments, “live C. perfringens” does not include attenuated CP.

The term “avian species” as used herein refers to all avian species. In an embodiment of the present disclosure, the bird is poultry such as a fowl (for example, a chicken, a turkey or a quail), a waterfowl (for example, a domestic duck or a domestic goose), a pigeon, a dove or a pheasant. It is an embodiment that the bird is a chicken or a turkey (e.g., a chick or poult). For in ovo delivery, the formulation is delivered to an egg at an embryonic stage of development of the subject of the avian species.

The term “immunologically effective amount” of a composition or vaccine of the disclosure comprising a live C. perfringens, is a quantity sufficient to, when administered to a subject, elicit an immune response to the C. perfringens and/or induce protective immunity, including homologous immunity and/or heterologous immunity, in the subject.

The term “homologous protection” as used herein with regard to C. perfringens, refers to protection against a same C. perfringens strain e.g. of the same genotype, for example, administration of a composition comprising a live C. perfringens strain, provides recipients who have successfully mounted an immune response, immune protection from infection by other C. perfringens strains having the same genotype (e.g. homologous protection).

The term “heterologous protection” as used herein with regard to C. perfringens, refers to protection against C. perfringens related strains, for example administration of a composition comprising a live C. perfringens strain, provides recipients who have successfully mounted an immune response, immune protection from infection by C. perfringens having the same genotype as well as by C. perfringens having different genotypes (e.g., heterologous protection).

As used herein, the term “CpG-ODN” refers to a strand of single-stranded synthetic nucleic acid molecule comprising at least one cytosine triphosphate deoxynucleotide followed by a guanine triphosphate deoxynucleotide motif connected through a phosphorothioate linkage or equivalent functional group excluding phosphodiester and naturally occurring groups, wherein the CpG is unmethylated. The strand can be between 6 to 55, for example between 12 to 24, or between 18 to 24, nucleotides long. For example, the nucleic acid molecule can be 6, 7, 8, 9, 10, 11, 12, 13, 14, 15, 16, 17, 18, 19, 20, 21, 22, 23, or 24 nucleotides long or longer. All three classes of CpG-ODN are encompassed, class A, class B and C. Also encompassed are hybrid structures comprising CpG-ODN nucleic acid molecules. Class B CpG-ODNs include a phosphorothioate backbone and one or more CpG dinucleotides, but no poly G motifs. Encompassed by this term are related Class B CpG-ODNs including ODN 2006, ODN 2007, ODN 1668, ODN 1862, ODN BW006, and ODN D-SL01. Class C CpG-ODNs include a phosphorothioate backbone, one or more CpG dinucleotides and a CpG-containing palindromic motif.

As used herein “CpG-ODN 2007” refers to a oligonucleotide of at least 14 nucleotides and up to 22 nucleotides, and comprising the sequence TCGTCGTTGTCGTT (SEQ ID NO: 2), optionally a 22-mer having the sequence 5′-TCG TCG TTG TCG TTT TGT CGT T-3′ (SEQ ID NO: 1) or any part thereof comprising TCGTCGTTGTCGTT (SEQ ID NO: 2) having a phosphorothioate backbone.

As used herein “cfu” refers to the concentration colony forming unit per mL.

As used herein, “humidity” refers to a quantity representing the amount of water vapour in the atmosphere or a gas, optionally in a chamber or housing.

As used herein, “humidex” or “humidity index” refers to a dimensionless index based on the dew point that describes the perceived temperature of a subject based on the temperature and humidity. The humidex is calculated according to the following formula:

$$H = {T}_{air} + 0.5555 \{ 6.11 \times exp [ 5417.753 ( \frac{1}{273.15} - \frac{1}{273.15 \times {T}_{dew}} ) ] - 10 \}$$

wherein H is humidity, Tₐᵢᵣ is the air temperature in ° C.; and T_{dew} is the dewpoint in ° C. 5417.7530 is a rounded constant based on the molecular weight of water, latent heat of evaporation, and the universal gas constant.

### Methods

An aspect of the disclosure includes a method of eliciting an immune response in one or more subject(s) against Clostridium perfringens (C. perfringens) infection and/or necrotic enteritis, the method comprising administering to one or more subject(s) an amount of a composition comprising live C. perfringens, and optionally a pharmaceutically acceptable carrier or diluent.

Another aspect of the disclosure includes a method of inducing protective immunity against or preventing Clostridium perfringens (C. perfringens) infection and/or necrotic enteritis, the method comprising administering to one or more subject(s) an amount of a composition comprising live C. perfringens, and optionally a pharmaceutically acceptable carrier or diluent.

In some embodiments, the pharmaceutically acceptable carrier diluent is water or saline. Suitable carriers and/or pharmaceutically acceptable carriers include for example water, including sterile water, or saline and may be added. Pharmaceutically acceptable carriers include for example carriers that are suitable for animal administration, for example phosphate buffered saline (PBS) or those which have been filtered for sterility.

In some embodiments, the composition is formulated for intrapulmonary (IPL) administration. In some embodiments, the composition is formulated for administration via nebulization. For example, the composition is an IPL vaccine composition formulated for IPL delivery. In some embodiments, the composition is no more viscous than water or distilled water, which has a viscosity of about 0.9 centipoise at 25° C. In some embodiments the composition is a liquid formulation and has a viscosity of 0.9 centipoise, about 0.9 centipoise at 25° C., or less than about 0.9 centipoise at 25° C. In some embodiments, the composition is no more viscous than PBS, which has a viscosity of about 1 centipoise at 25° C. In some embodiments the composition is a liquid formulation and has a viscosity of 1 centipoise at 25° C., about 1 centipoise at 25° C., or less than about 1 centipoise at 25° C.

In some embodiments, the composition is formulated as or to produce microdroplets, for example when nebulized. In some embodiments, the microdroplets have a particle size of about 0.5 μm to about 5 μm. As described herein, intrapulmonary (IPL) delivery of live C. perfringens, can be used to protect broiler chicks from C. perfringens infection and/or necrotic enteritis. As also demonstrated herein, IPL microdroplets with a size 0.5 μm-5 μm, which with inhalation, goes deep down into the lungs. Normal practice for delivering an aerosol vaccine to subjects would target a shallower delivery, with the vaccine entering the respiratory system at the nostril (14). Sending antigens deep down into the trachea and lungs is usually avoided because it would be expected that placing a live bacterium so directly into a subject's lung would cause them illness and death (14). IPL delivery comprises delivery of microdroplets having a particle size of less than 5 μm, as described herein, as opposed to aerosol vaccines or coarse spray vaccines which involve delivery of larger size particles (14). Surprisingly, the inventors have shown herein that IPL delivery of live C. perfringens into the subject lungs, does not induce disease and/or is not fatal but rather induces protective immunity.

In some embodiments, the amount of live C. perfringens delivered or to be delivered per subject is between about 1×10⁶ bacteria to about 1×10⁹ bacteria. In some embodiments, the amount of live C. perfringens delivered or to be delivered per subject is between about 1×10⁷ to about 1×10⁹ bacteria. In some embodiments, the amount of live C. perfringens delivered or to be delivered per subject is about 1×10⁶ bacteria. In some embodiments, the amount of live C. perfringens delivered or to be delivered per subject is about 1×10⁸ bacteria. In some embodiments, the amount of live C. perfringens is about 1×10⁷ bacteria. Even if subjects, for example poultry, ingest the composition during nebulization, the dose as demonstrated herein does not cause necrotic enteritis.

In some embodiments, the concentration of the composition is about 0.5×10⁷ cfu to about 1.6×10¹⁰ cfu. In some embodiments, the concentration of the composition is about 0.8×10⁷ cfu. In some embodiments, the concentration of the composition is about 1×10⁷ cfu. In some embodiments, the concentration of the composition is about 1.6×10⁷ cfu. In some embodiments, the concentration of the composition is about 0.8×10⁹ cfu. In some embodiments, the concentration of the composition is about 1×10⁹ cfu. In some embodiments, the concentration of the composition is about 1.6×10⁹ cfu.

In some embodiments, the amount of live C. perfringens delivered or to be delivered per subject is about 1×10⁸ bacteria.

The composition can be delivered in a nebulization chamber. For example, if the per subject dose of about 1×10⁶ bacteria to about 1×10⁸ bacteria is desired, a chamber concentration of about 1.1×10⁸ bacteria/m³ to about 1.1×10¹⁰ bacteria/m³ can be used. A chamber size of 0.036 m³ was used herein to deliver 4-5 mL of the composition comprising the live C. perfringens to 40 chicks. In an example where the composition is to be delivered to 8,000 chicks, 500 mL to 1 L of the composition comprising the live C. perfringens may be used and a chamber for example a commercial-scale poultry nebulizer as described in Goonewardene et al., (13) may be used.

In some embodiments, the composition is administered to the one or more subject(s) for between about 15 to about 30 minutes. In some embodiments, the composition is administered to the one or more subject(s) for about 30 minutes. In some embodiments, the composition is administered to the one or more subject(s) for about 15 minutes. In some embodiments, the composition is administered to the one or more subject(s) for about 20 minutes. In some embodiments, the composition is administered to the one or more subject(s) for about 25 minutes.

As demonstrated herein, the vaccine can provide heterologous protection. For example, it is demonstrated that the composition comprising live CP strain containing 3 toxin genes provided heterologous protection to challenge with CP strains having 4 toxin genes.

The composition in some embodiments comprises live C. perfringens with at least one toxin gene, at least two toxin genes, at least three toxin genes or at least four toxin genes. In some embodiments, the composition comprises one or more than one live C. perfringens strains.

Nebulizers capable of providing a desired droplet size can be used. For example a commercial-scale poultry nebulizer as described in Goonewardene et al., (13), a chamber nebulizer delivery system (Kent Scientific Corporation, Connecticut), a Rcom Bird Brooder (Incomax Tecnologia Na Criacso, Portugal), or a chick nebulizing system (RMD Group of Companies, Saskatchewan) may be used. For example, nebulizers that produce microdroplets that have a particle size of about 0.5 μm to about 5 μm can be used. As mentioned, the size of the microdroplets allows the composition to enter deep into the subject lungs.

In a further embodiment, the composition is administered in an environment (e.g. a chamber) wherein the humidity is less than about 70%, less than about 65% or less than about 60%, optionally between about 40% and about 70%, preferably between about 40% and about 60%.

In a preferred embodiment, the one or more subject(s) is administered the composition in an environment (e.g., a chamber) where the humidex is, below or about 28, or below or about 27 or below or about 26. The humidex is for example 25 or greater. In some embodiments, the humidex is between 25 to 28.

In some embodiments, the composition is administered in an environment (e.g. a chamber) having a temperature of about 25° C. to about 30° C. In some embodiments, the composition is administered in an environment (e.g., a chamber) having a temperature of about 28° C. to about 30° C. In some embodiments, the composition is administered in an environment (e.g., a chamber) having a temperature of about 25° C., about 26° C., about 27° C., about 28° C., about 29° C. or about 30° C.

In some embodiments, the composition is administered prior to exposure to C. perfringens. For example, the composition can be administered in the hatchery and before the subjects are moved to barns or mixed with older subjects.

In some embodiments, the composition is lyophilized, for example, the composition that is lyophilized is reconstituted or resuspended in a suitable diluent, prior to administration.

In some embodiments, the composition further comprises a disaccharide.

In some embodiments, the disaccharide is sucrose or mannose.

In some embodiments, the composition lacks skim milk.

In some embodiments, the lyophilized composition is resuspended in a suitable diluent such as saline or water, prior to administration to the one or more subject(s).

In some embodiments, the lyophilized composition is resuspended in saline prior to administration to the one or more subject(s). In some embodiments, the lyophilized composition is resuspended in phosphate buffered saline (PBS) prior to administration to the one or more subject(s). In some embodiments, the lyophilized composition is resuspended in water, optionally distilled water, prior to administration to the one or more subject(s).

The inventors have discovered for example that their intrapulmonary live vaccination technique for delivering live C. perfringens can mount a strong systemic immune response (e.g., in the body) as well as mucosal immune responses (e.g., in the intestine) against necrotic enteritis in the intestine without an adjuvant.

In some embodiments, the composition is sterile, which as used herein refers to the composition having no other bacteria other than C. perfringens.

In some embodiments, the one or more subject(s) is/are poultry. In some embodiments, the poultry is a duck, chicken, or turkey. In some embodiments, the one or more subject(s) is/are a chicken. In some embodiments, the one or more subject(s) is/are a broiler chicken.

In some embodiments, the composition is administered within about 24 to about 36 hours of hatch, for example within about 24 hours, within about 28 hours, within about 32 hours or within about 36 hours of hatch. In some embodiments, the composition is administered within about 24 hours of hatch.

In some embodiments, the administration to the one or more subject(s) is performed in a closed chamber or chicken enclosure unit, such as nebulizer chamber described in Goonewardene et al., (13), a chamber nebulizer delivery system (Kent Scientific Corporation, Connecticut), a Rcom Bird Brooder (Incomax Tecnologia Na Criação, Portugal) or a chick nebulizing system (RMD Group of Companies, Saskatchewan).

In some embodiments, one or more CpG oligodeoxynucleotide(s) (CpG-ODNs) are administered prior to the initial administration of the composition. In some embodiments, the one or more CpG-ODNs are administered prior to hatch. In some embodiments, the one or more CpG-ODNs are administered via the in ovo route. In some embodiments, where the subject is a chicken species, the CpG-ODNs are administered in ovo at about day 18 of incubation. The incubation period for chicken eggs is about 20 to about 21 days and can be up to 30 days for other poultry. For other avian species, the CpG-ODNs can be administered in ovo 3 days or 2 days prior to hatch. For example the CpG-ODNs can be CpG-ODN (TCGTCGTTGTCGTTTTGTCGTT, Class B, 2007) (SEQ ID NO: 1).

In some embodiments, the amount of CpG-ODNs is between about 30 μg/egg to about 60 μg/egg. In some embodiments, the amount of CpG-ODNs is between about 30 μg/egg to about 50 μg/egg. The CpG-ODNs can be provided as a liquid composition suitable for in ovo administration. In some embodiments, the CpG-ODNs are diluted in water, distilled water, saline and/or PBS.

In some embodiments, the one or more CpG-ODN(s) are administered using intrapulmonary delivery. In some embodiments, the one or more CpG-ODN(s) is administered with the composition simultaneously. In some embodiments, the composition comprises one or more CpG-ODN(s). In some embodiments, the one or more CpG-ODN(s) is an inhalable CpG-ODN, such as AZD1419.

In some embodiments, a booster is administered after the initial administration of the composition. In some embodiments, the booster is a composition comprising inactivated C. perfringens. In other embodiments, the booster is recombinant CP toxin genes. In some embodiments, the booster is administered subcutaneously.

In an embodiment, the composition is comprised in a quantity sufficient to immunize at least 1, at least 10, at least 20, at least 30, at least 50, at least 75 and/or at least 100 subjects. In another embodiment, the composition and/or vaccine is comprised in a quantity sufficient to immunize at least 500, at least 1,000, at least 2,000, at least 3,000, at least 4,000, at least 5,000, at least 6,000, at least 7,000, at least 8,000, at least 9,000 or at least 10,000 subjects.

### Compositions/Compositions for Use

Another aspect of the disclosure includes a composition comprising live Clostridium perfringens (C. perfringens), and optionally a pharmaceutically acceptable carrier or diluent for use in eliciting an immune response in one or more subject(s) against C. perfringens infection and/or necrotic enteritis and/or inducing protective immunity against or preventing C. perfringens infection and/or necrotic enteritis.

Another aspect of the disclosure includes a single dose composition comprising live Clostridium perfringens (C. perfringens), and optionally a pharmaceutically acceptable carrier or diluent. The diluent can comprise water such as distilled water, or saline such as PBS. The composition or container comprising the composition can be sterile.

In an embodiment, the composition is an intrapulmonary vaccine composition.

In some embodiments, the composition is formulated for intrapulmonary administration. The composition can be provided in a container, optionally a sterile container to provide a plurality of dosages.

The composition in some embodiments comprises live C. perfringens with at least one toxin gene, at least two toxin genes, at least three toxin genes or at least four toxin genes. In some embodiments, the composition comprise one or more than one live C. perfringens strains.

In some embodiments, the dosage per subject is about 1×10⁶ bacteria to about 1×10⁹ bacteria of the live C. perfringens. In some embodiments, the dosage per subject is about 1×10⁷ bacteria to about 1×10⁹ bacteria of the live C. perfringens. In some embodiments, the dosage per subject is about 1×10⁶ bacteria of the live C. perfringens. In some embodiments, the dosage per subject is about 1×10⁸ bacteria of the live C. perfringens.

In some embodiments, the concentration of the composition is about 0.5×10⁷ cfu to about 1.6×10¹⁰ cfu. In some embodiments, the concentration of the composition is about 1×10⁷. In some embodiments, the concentration of the composition is about 1×10⁹. In some embodiments, the concentration of the composition is about 0.8×10⁷ cfu. In some embodiments, the concentration of the composition is about 0.8×10⁹ cfu. In some embodiments, the concentration of the composition is about 1.6×10⁷ cfu. In some embodiments, the concentration of the composition is about 1.6×10⁹ cfu.

In some embodiments, the composition is formulated for administration via nebulization. For example, the composition is an IPL vaccine composition formulated for IPL delivery.

In some embodiments, the composition is formulated as or to be delivered as microdroplets, for example when nebulized.

In some embodiments, the microdroplets have a particle size of about 0.5 μm to about 5 μm.

In some embodiments, the dosage of live C. perfringens is between about 1×10⁶ bacteria to about 1×10⁹ bacteria per subject, optionally at about 1×10⁸ bacteria per subject.

In some embodiments, the dosage of live C. perfringens is about 1×10⁸ bacteria per subject. In some embodiments, the dosage of live C. perfringens is about 1×10⁶ bacteria.

In some embodiments, the composition is for administration to the one or more subject(s) for about 30 minutes.

In some embodiments, the composition is for administration in an environment (e.g., a chamber) having a temperature of about 28° C. to about 30° C.

In some embodiments, the composition is for administration in an environment (e.g., a chamber) having less than about 70% humidity, optionally between about 40% and about 70% humidity, preferably between about 40% and about 60% humidity.

In a preferred embodiment, the composition is for administration in an environment (e.g., a chamber) where the humidex is, below or about 28, or below or about 27 or below or about 26. The humidex is for example 25 or greater. In some embodiments, the humidex is between 25 to 28.

In some embodiments, one or more CpG oligodeoxynucleotide(s) is/are for administration prior to administration of the composition. In some embodiments, where the subject is a chicken species, the one or more CpG-ODN(s) is for administration in ovo, optionally at about day 18 of incubation. The incubation period for chicken eggs is about 20 to about 21 days and can be up to 30 days for other poultry. For other avian species, the CpG-ODN(s) can be for administration in ovo 3 days or 2 days prior to hatch. For example the CpG-ODN(s) can be CpG-ODN (TCGTCGTTGTCGTTTTGTCGTT, Class B, 2007) (SEQ ID NO: 1).

In some embodiments, the amount of CpG-ODN(s) is between about 30 μg/egg to about 60 μg/egg. In some embodiments, the amount of CpG-ODN(s) is about 50 μg/egg.

The CpG-ODNs may be provided as a liquid composition. In some embodiments, the one or more CpG-ODN(s) is/are diluted/reconstituted in water, such as distilled water, or saline such as phosphate buffered saline (PBS).

In some embodiments, the one or more CpG-ODN(s) are administered using intrapulmonary delivery. In some embodiments, the one or more CpG-ODN(s) is administered with the composition simultaneously. In some embodiments, the composition comprises one or more CpG-ODN(s). In some embodiments, the one or more CpG-ODN(s) is an inhalable CpG-ODN, such asAZD1419.

In some embodiments, the composition is for administration prior to exposure to C. perfringens. For example, the composition can be for administration in the hatchery and before the subjects are moved to barns or mixed with older subjects.

In some embodiments, the composition is for administration at least 20 days to at least 22 days prior to exposure to C. perfringens.

In some embodiments, the composition is lyophilized. For example, the composition that is lyophilized is reconstituted or resuspended in a suitable diluent prior to administration.

In some embodiments, the composition further comprises a disaccharide.

In some embodiments, the disaccharide is sucrose or mannose.

In some embodiments, the composition lacks skim milk. For example, the composition constituents as shown in the example are used to provide a powdery product after lyophilization which allowed for better reconstitution.

In some embodiments, the lyophilized composition is resuspended in a suitable diluent such as saline or water.

In some embodiments, the lyophilized composition is resuspended in saline.

In some embodiments, the composition further comprises an adjuvant, particularly in a booster composition.

In some embodiments, the adjuvant comprises CpG oligodeoxynucleotides.

In some embodiments, the composition is sterile.

In some embodiments, the one or more subject(s) is of the avian species.

In some embodiments, the one or more subject(s) is/are poultry.

In some embodiments, the one or more subject(s) is/are a chicken.

In some embodiments, the composition is for administration within about 24 to about 36 hours of hatch, for example within about 24 hours, within about 28 hours, within about 32 hours or within about 36 hours of hatch. In some embodiments, the composition is for administration within about 24 hours of hatch.

In some embodiments, the composition is for administration to the one or more subject(s) in a closed chamber.

Conventional procedures and ingredients for the selection and preparation of suitable compositions are described, for example, in Remington's Pharmaceutical Sciences (2000—20th edition) and in The United States Pharmacopeia: The National Formulary (USP 24 NF19) published in 1999.

The compositions described herein can be prepared by per se known methods for the preparation of pharmaceutically acceptable compositions that can be administered to subjects such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the subject.

The dosage of compositions of the present disclosure can vary depending on many factors such as the pharmacodynamic properties of the composition, the mode of administration, the age, health and weight of the recipient, the nature and extent of the symptoms, the frequency of the treatment and the type of concurrent treatment, if any, and the clearance rate of the composition in the subject to be treated. One of skill in the art can determine the appropriate dosage based on, for example the above factors. Compositions of the present disclosure may be administered initially in a suitable dosage that may be adjusted as required, depending on the clinical response, and may be administered in a single daily dose or the total daily dose may be divided into two, three or four daily doses.

### Uses

Another aspect of the disclosure includes use of a composition comprising live Clostridium perfringens (C. perfringens), and optionally a pharmaceutically acceptable carrier or diluent for eliciting an immune response in one or more subject(s) against C. perfringens infection and/or necrotic enteritis and/or for inducing protective immunity against or preventing C. perfringens infection and/or necrotic enteritis.

Another aspect of the disclosure includes use of a composition comprising live Clostridium perfringens (C. perfringens), and optionally a pharmaceutically acceptable carrier or diluent, in the manufacture of a medicament for eliciting an immune response in one or more subject(s) against C. perfringens infection and/or necrotic enteritis and/or for inducing protective immunity against or preventing C. perfringens infection and/or necrotic enteritis.

In an embodiment, the composition is an intrapulmonary vaccine composition.

The diluent can comprise water such as distilled water, or saline such as PBS. The composition or container comprising the composition can be sterile.

In some embodiments, the composition is formulated for intrapulmonary administration. The composition can be provided in a container, optionally a sterile container to provide a plurality of dosages.

In some embodiments, the dosage per subject is about 1×10⁶ bacteria to about 1×10⁹ bacteria of the live C. perfringens. In some embodiments, the dosage per subject is about 1×10⁸ bacteria of the live C. perfringens. In some embodiments, the dosage per subject is about 1×10⁷ bacteria of the live C. perfringens. In some embodiments, the dosage per subject is about 1×10⁶ bacteria of the live C. perfringens.

In some embodiments, the concentration of the composition is about 0.5×10⁷ cfu to about 1.6×10¹⁰ cfu. In some embodiments, the concentration of the composition is about 1×10⁷ cfu. In some embodiments, the concentration of the composition is about 1×10⁹ cfu. In some embodiments, the concentration of the composition is about 0.8×10⁹ cfu. In some embodiments, the concentration of the composition is about 0.8×10⁷ cfu. In some embodiments, the concentration of the composition is about 1.6×10⁹ cfu. In some embodiments, the concentration of the composition is about 1.6×10⁷ cfu.

In some embodiments, the composition is formulated for administration via nebulization. For example, the composition is an IPL vaccine composition formulated for IPL delivery.

In some embodiments, the composition is formulated as or to be delivered as microdroplets, for example when nebulized.

In some embodiments, the microdroplets have a particle size of about 0.5 μm to about 5 μm.

In some embodiments, the concentration of the composition is between about 0.5×10⁷ cfu to about 1.6×10¹⁰ cfu.

In some embodiments, the concentration of the composition is about 1×10⁹ cfu.

In some embodiments, the composition is for administration to the one or more subject(s) for about 30 minutes.

In some embodiments, the composition is for administration in an environment (e.g. a chamber) having a temperature of about 28° C. to about 30° C.

In some embodiments, the composition is for administration in an environment (e.g. a chamber) having less than about 70% humidity, optionally between about 40% and about 70% humidity, preferably between about 40% and about 60% humidity.

In a preferred embodiment, the composition is for administration in an environment (e.g., a chamber) where the humidex is, below or about 28, or below or about 27 or below or about 26. The humidex is for example 25 or greater. In some embodiments, the humidex is between 25 to 28.

In some embodiments, one or more CpG oligodeoxynucleotide(s) is/are for administration prior to administration of the composition.

In some embodiments, where the subject is a chicken species, the one or more CpG-ODN(s) is for administration in ovo, optionally at about day 18 of incubation. The incubation period for chicken eggs is about 20 to about 21 days and can be up to 30 days for other poultry. For other avian species, the CpG-ODNs can be for administration in ovo 3 days or 2 days prior to hatch. For example, the CpG-ODNs can be CpG-ODN (TCGTCGTTGTCGTTTTGTCGTT, Class B, 2007) (SEQ ID NO: 1). In some embodiments, the amount of CpG-ODNs is between about 30 μg/egg to about 60 μg/egg. In some embodiments, the amount of CpG-ODNs is about 50 μg/egg.

In some embodiments, the one or more CpG-ODN(s) are diluted or reconstituted in water, such as distilled water, or saline such as PBS.

In some embodiments, the one or more CpG-ODN(s) are administered using intrapulmonary delivery. In some embodiments, the one or more CpG-ODN(s) is administered with the composition simultaneously. In some embodiments, the composition comprises one or more CpG-ODN(s). In some embodiments, the one or more CpG-ODN(s) is an inhalable CpG-ODN, such asAZD1419.

In some embodiments, the composition is for administration prior to exposure to C. perfringens. For example, the composition can be for administration in the hatchery and before the subjects are moved to barns or mixed with older subjects.

In some embodiments, the composition is for administration at least 20 days to at least 22 days prior to exposure to C. perfringens.

In some embodiments, the composition is lyophilized. For example, the composition that is lyophilized is reconstituted or resuspended in a suitable diluent prior to administration.

In some embodiments, the composition further comprises a disaccharide.

In some embodiments, the disaccharide is sucrose or mannose.

In some embodiments, the composition lacks skim milk.

In some embodiments, the lyophilized composition is resuspended in a suitable diluent such as saline or water.

In some embodiments, the lyophilized composition is resuspended in saline.

In some embodiments, the composition further comprises an adjuvant.

In some embodiments, the adjuvant comprises CpG oligodeoxynucleotides.

In some embodiments, the composition is sterile.

In some embodiments, the one or more subject(s) is of the avian species.

In some embodiments, the one or more subject(s) is/are poultry.

In some embodiments, the one or more subject(s) is/are a chicken.

In some embodiments, the composition is for administration within about 24 to about 36 hours of hatch, for example within about 24 hours, within about 28 hours, within about 32 hours, or within about 36 hours of hatch. In some embodiments, the composition is for administration within about 24 hours of hatch.

In some embodiments, the composition is for administration to the one or more subject(s) in a closed chamber.

### Kits

An aspect of the disclosure includes a kit comprising at least any of the compositions described herein, optionally separately provided. For example, the kit can comprise a composition described herein, in combination with or separate from CpG-ODNs and/or a booster composition.

In some embodiments, the kit further comprises instructions for use. In some embodiments, the kit further comprises one or more vials, containers, or other suitable packaging. In some embodiments, the kit further comprises a nebulizer.

In some embodiments, the kit comprises lyophilized compositions described herein, for example the composition comprising live CP, and optionally a composition comprising CpG-ODNs and/or booster. In some embodiments, the kit further comprises a diluent for use in reconstituting the lyophilized composition. In some embodiments, the diluent is saline. The kit can also comprise CpG-ODN, (optionally in solution or for example formulated for in ovo delivery or intrapulmonary delivery). The kit can also comprise a syringe in some embodiments. In some embodiments, the kit further comprises a chamber or chicken enclosure unit.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### EXAMPLES

### Example 1

### Materials and Methods:

Delivery of C. perfringens (CP) Vaccine Following Immune Modulation with CpG-ODN

The objectives of this experiment were to investigate protection and mucosal IgA production following intrapulmonary (IPL) delivery of a CP vaccine (live and inactivated) in broilers against CP, and to investigate immunomodulatory effect of in ovo delivery of CpG-ODN on the CP vaccine. CpG-ODN 2007 (SEQ ID NO: 1) was used in all formulations including CpG-ODNs in this example.

### Antigen Preparation for Nebulization:

CP was cultured in 50 mL of fluid thioglycollate medium for 16 hr in 100 mL capacity Erlenmeyer flasks under anaerobic conditions (BD Gas Pak EZ chamber with 2 sachets of AnaeroGen 3.5 L, Thermo scientific) at 37° C. The following day, 0.04% formaldehyde was added to the culture and incubated at 37° C. for overnight. To ensure inactivation of CP, 100 μL of overnight culture was plated on 5% Columbia sheep blood agar in duplicate. The inactivated culture solution was dispensed in 50 mL centrifuge tubes in 35 mL aliquots and centrifuged (Sorvall Legend RT, Mandel) at 4,000 rpm for 20 min. Bacterial pellets were transferred to small vaccine vials with 500 μL of 10% sucrose and stored at −80° C. overnight. The following day, lyophilization was performed in the lyophilizer for 12 hr. For live antigen preparation, all the above steps were the same except for the formaldehyde inactivation. To confirmation viability of CP following lyophilization, lyophilized product was cultured on 5% Columbia sheep blood agar in duplicate.

### Experiment Design:

Groups of embryonated eggs from Ross broiler breeders were incubated in an incubator in ACU, University of Saskatchewan. Embryonated eggs were candled at day 11 and 18 of incubation to confirm viability. At day 18 of incubation, viable eggs were randomly divided into eight groups (25 eggs/group). Group numbers 3 and 7 received 100 μL CpG-ODN (50 μg/egg) by the in ovo route using 23G×1 inch needle (Table 4). Melted wax was used to seal the injection site following in ovo administration. At day of hatch, chicks in group numbers 3 and 4 were nebulized with live CP (approximately 1×10⁸ cfu/bird). Groups 5 and 6 were nebulized with inactivated CP [approximately 1×10⁸ cfu/bird+CpG-ODN (10 μg/bird)]. Sections of lungs were collected (3 birds/group) at 24, 48, and 72 hr post-administration of CP (IPL) for microscopic evaluation of pulmonary parenchyma. These samples were preserved in 10% neutral buffered formalin. CP challenge was conducted as previously described, briefly, 15 hr culture, feed (28% protein) was mixed with fluid thioglycollate media (1:1) and fed twice daily for three days. CP was aerosolized as microdroplets (particle size of 0.5-5 μm) by using a Compressor Nebulizer.

### CP Challenge Preparation:

CP was grown in cooked meat medium (Sigma-Aldrich) for 24 h at 37° C. under anaerobic conditions (BD Gas Pak EZ chamber with 2 sachets of AnaeroGen 3.5 L, Thermo scientific). Cooked meat medium was added to Thioglycollate medium (Sigma-Aldrich) at 3% (v/v). Cooked meat medium culture was incubated anaerobically at 37° C. for 15 h. The bacterial growth at 15 h culture had approximately 1×10⁹ CFU/mL. The fluid Thioglycollate medium-grown culture was then mixed with feed at a ratio of 1:1 (v/w) for groups 1 and 2. Feed was withdrawn prior to exposure of birds to CP. CP was administered twice daily in-feed for 3 consecutive days.

Detection of IgA and IgY Against C. perfringens

The indirect ELISA was developed and used. The indirect ELISA was developed to detect serum (IgY) and mucosal (IgA) antibody responses against C. perfringens whole cell antigen. The test was optimized using checkerboard titrations of C. perfringens whole cell antigen. The OD values were determined for test samples after subtracting the negative control OD values. The samples were tested as follows:

Ninety-six well microtiter plates were coated with heat-inactivated C. perfringens (0.5 OD) and kept at 4° C. overnight. Coated wells were blocked by the addition of 100 ul of 10% skim milk diluted in phosphate-buffered saline (pH−7.4) with incubation for 30 minutes at room temperature. Blocked plates were then washed four times with distilled water. Sera were tested at 1:100 dilutions and mucosal scrapings at 1:10 dilution, in duplicates. The serum and mucosal supernatant from each bird were separately diluted in 1% skim milk diluted in PBST (pH −7.4). Then plates were washed 6× with distilled water and then added 100 μL of goat anti-chicken IgA (Gene tex) (1:2000) and incubated 2 hrs at room temperature. Plates were washed 6× with distilled water and added 100 μL of 3,3′,5,5′-Tetramethylbenzidine (TMB substrate, Molecular innovations, Cedarlane) and incubated for 30 minutes. Negative controls consisted of triplicate wells with all reagents except either antigen, or primary, or secondary antibody. The optical density value (OD) at 405 nm, was determined using SpectraMax Plus 340 PC Microplate Reader. OD values for each bird were calculated as the average of OD405 values from duplicate wells minus the average OD405 values of the negative controls.

### Nebulization of CP Vaccine

Live or inactivated CP vaccine was aerosolized as micro-droplets (particle size of 0.5-5 μm) using a Compressor Nebulizer (705-470) unit (AMG Medical Inc; Montreal, QC, Canada). CP vaccine was aerosolized in a closed 0.036 m³ acrylic chamber for 30 min. The control group of birds was aerosolized with saline for 30 min in the acrylic chamber using the Compressor Nebulizer. The temperature was maintained at 28-30° C. in the acrylic chamber during administration of vaccine or saline or saline as we previously described for CpG-ODN (Avian Dis. 2017 December; 61(4):503-511). CP challenge was done for 3 consecutive days (CP mixed with feed) between 20-23 days of age. IPL vaccination was done 20 days prior to challenge.

### Statistical Analysis

Histopathological lesion scores were analyzed using Graph Pad Prism 6 (95% of Confidence interval). Tukey's multiple comparison test was performed after one way ANOVA to compare the difference between groups.

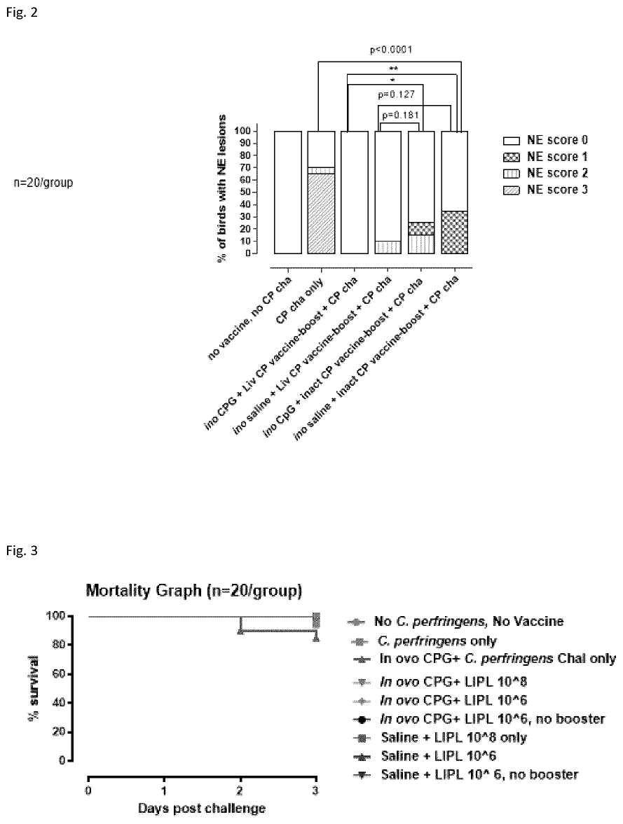

TABLE 1
Study design of animal experiment (results shown in FIG. 2).

| | | | | | CP |
|---|---|---|---|---|---|
| | No. | In ovo @ | | | Challenge |
| Group | of | day 18 | | Booster @ | (day20- |
| # | birds | incubation | IPL at hatch | day 10 | 21-22) |
| 1 | 20 | Saline | − | − | − |
| 2 | 20 | Saline | − | − | + |
| | | (100 μl) | | | |
| 3 | 20 | CpG-ODN | Live C. | 1 × 10⁸cfu | + |
| | | (50 μg)/ | perfringens | CP with | |
| | | egg | (1 × 10⁸) | CpG-ODN | |
| | | | | (10 μg/bird) | |
| 4 | 20 | − | Live C. | 1 × 10⁸cfu | + |
| | | | perfringens | CP with | |
| | | | (1 × 10⁸) | CpG-ODN | |
| | | | | (10 μg/bird) | |
| 5 | 20 | CpG-ODN | CpG (10 μg) with | 1 × 10⁸cfu | + |
| | | (50 μg)/ | inactivated C. | CP with | |
| | | egg | perfringens | CpG-ODN | |
| | | | (1 × 10⁸) | (10 μg/bird) | |
| 6 | 20 | − | CpG (10 μg) with | 1 × 10⁸cfu | + |
| | | | inactivated C. | CP with | |
| | | | perfringens | CpG-ODN | |
| | | | (1 × 10⁸) | (10 μg/bird) | |

The following is a description of groups involved in the animal experiment for which results are shown in FIG. 2. The objective of this experiment was to demonstrate delivery live or inactivated OP vaccine by the IPL route against NE.
Group 1: This group is a control group with no intervention.
Group 2: This group is a positive control for necrotic enteritis (NE), Broilers were challenged with Clostridium perfringens (OP). This group is to compare with all other groups to study challenge protection against OP.
Group 3: Eggs received 50 μg CpG-ODN by the in ovo route at day 18 of incubation to modulate the immune system before vaccination at the time of hatch. At hatch, birds were vaccinated with live OP (1×10⁸ cfu) with no adjuvant by the IPL route and received an inactivated CP (1×10⁸ cfu) booster with 10 μg CpG-ODN as an adjuvant by the SC route at day 10 of age.
Group 4: Eggs received saline by the in ovo route. At hatch, birds were vaccinated with live CP (1×10⁸ cfu) with no adjuvant by the IPL route and received an inactivated CP (1×10⁸ cfu) booster with 10 μg CpG-ODN as an adjuvant by the SC route at day 10 of age.
Group 5: Eggs received 50 μg CpG-ODN by the in ovo route at day 18 of incubation to modulate the immune system before vaccination at the time of hatch. At hatch, birds were vaccinated with inactivated CP (1×10⁸ cfu) with no adjuvant by the IPL route and boostered with 10 μg CpG-ODN as an adjuvant by the SC route at day 10 of age.
Group 6: Eggs received saline by the in ovo route at day 18 of incubation. At hatch, birds were vaccinated with inactivated CP (1×10⁸ cfu) and boostered with 10 μg CpG-ODN as an adjuvant by the SC route at day 10 of age.

Conclusions: Groups vaccinated with live by the IPL route induced protective immunity against NE. The group vaccinated with live CP following in ovo delivery of CpG-ODN had superior protection against NE (100%).

TABLE 2
Assessment of Histopathological lesions

| Score | Description |
|---|---|
| 0 | No lesions |
| 1 | Necrosis of intestinal villi; focal (1-2) acute |
| 2 | Necrosis of intestinal villi; multifocal to |
| | coalescing, acute |
| 3 | Necrosis of intestinal villi; diffuse, acute, severe |

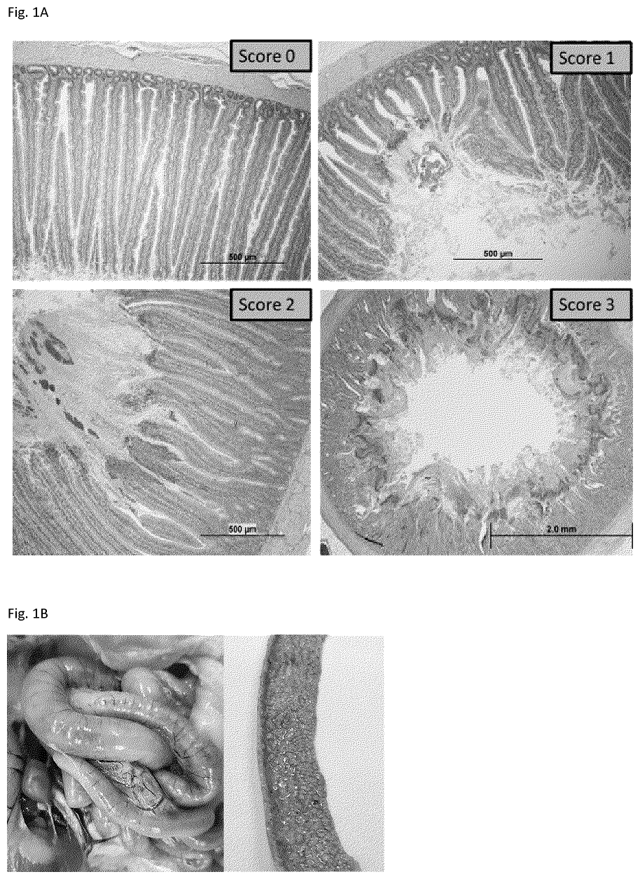

The results shown in FIG. 2 are discussed herein and the groups are identified in Table 1. Scoring for assessment of histopathological lesions is discussed in Table 2. Images of gross and histopathological lesions associated with NE in the animal models described herein are shown in FIGS. 1A-1B. There were no NE lesions observed in the group with no vaccine and no CP challenge. The group with no vaccine and challenged with CP had 5% of birds with gross lesions and 60% of birds with histopathological lesions of NE. There were no gross or histopathological lesions (0%) in the group vaccinated with live CP vaccine by the IPL route following in ovo CpG-ODN delivery while group received live CP vaccine by the IPL route with no in ovo CpG-ODN had 10% birds with histopathological lesions. Group vaccinated with inactivated CP vaccine by the IPL route following in ovo CpG-ODN had 5% gross lesions and 20% histopathological lesions of NE while group vaccinated with inactivated CP vaccine by the IPL route with no CpG-ODN had 25% birds with histopathological lesions of NE. Broilers vaccinated with live or inactivated CP vaccine by the IPL route had a significantly lower level of NE lesions compared to the group not vaccinated with a CP vaccine but challenge with CP (P<0.0001). There was a significant difference between group ino CpG+ Live CP vaccine-boost+ CP cha when compared with ino CpG+inact CP vaccine-boost+CP cha (p=0.0471) and ino saline+inact CP vaccine-boost+CP cha (p=0.0083) respectively.

### Example 2

Live C. perfringens (CP) Vaccine Delivered by the Intrapulmonary (IPL) Route

The objectives of this experiment were to investigate immunoprotective effects of IPL delivery of a live CP vaccine at a lower dose following in ovo CpG-ODN administration, to investigate immunoprotective effects of IPL delivery of a live CP vaccination following in ovo CpG-ODN but without a subcutaneous (SC) boost at 10 days of age, to investigate immunoprotective effects of IPL delivery of a live CP vaccine with no in ovo CpG-ODN and without a subcutaneous (SC) boost at 10 days of age, and to investigate cellular mechanisms of immunoprotection of IPL delivery of live CP vaccine on B-cells, T-cells and macrophages. CpG-ODN 2007 (SEQ ID NO: 1) was used in all formulations including CpG-ODNs in this example.

### Materials and Methods:

### Experimental Design

This work was approved by the Animal Research Ethics Board (AREB), University of Saskatchewan and adhered to the guidelines of the Canadian Council on Animal Care. Eggs (n=600) were incubated at the ACU, WCVM, University of Saskatchewan. Eggs were candled at day 11 and 18 of incubation to confirm viability. At day 18 of incubation, fertile eggs were randomly assigned to nine groups (N=40 eggs per group). Incubating eggs were grouped as: 1) no CP vaccine and no CP challenge; 2) in ovo CpG-ODN and CP challenge; 3) CP challenge only; 4) in ovo CpG-ODN+live CP vaccine (1×10⁸ cfu/bird) and inactivated CP boost (1×10⁸ cfu/bird with 10 μg CpG-ODN)+CP challenge; 5) in ovo CpG-ODN+live CP vaccine (1×10⁶ cfu) and inactivated CP boost (1×10⁶ cfu/bird with 10 μg CpG-ODN)+CP challenge; 6) in ovo CpG-ODN+live CP vaccine (1×10⁶ cfu/bird), no CP boost+CP challenge; 7) in ovo saline+live CP vaccine (1×10⁸ cfu/bird) and CP boost (1×10⁸ cfu/bird with 10 μg CpG-ODN)+CP challenge; 8) in ovo saline+live CP vaccine (1×10⁶ cfu) and CP boost (1×10⁶ cfu/bird with 10 μg CpG-ODN)+CP challenge; 9) in ovo saline+live CP vaccine (1×10⁶ cfu), no CP boost+CP challenge. At day of hatch groups of chicks in respective groups were nebulized with lyophilized live CP (approximately 1×10⁸ or 1×10⁶ cfu/bird). The lung tissues (n=3) were collected at 24, 48, and 72 h post nebulization for histopathological evaluation. OP boost was prepared by inactivated OP (1×10⁸ or 1×10⁶ cfu/bird) adjuvanted with CpG-ODN (18 μg/bird) and delivered by the subcutaneous route (SC) at 10 days of age. All groups except group 1 were challenged in feed OP (1:1 ratio of 1×10⁹ cfu/g feed) at 20, 21 and 22 days of age. Additional groups of birds (n=10) were used to measure IgA and IgG and these birds were not challenge with OP and collected mucosal scrapings and blood at 25 days of age. Groups that designated as “in ovo CpG-ODN” received 50 μg of CpG-ODN in 100 μl into the amniotic cavity using a 23G, 1 inch needle. Melted wax was used to seal the in ovo injection site following CpG-ODN administration.

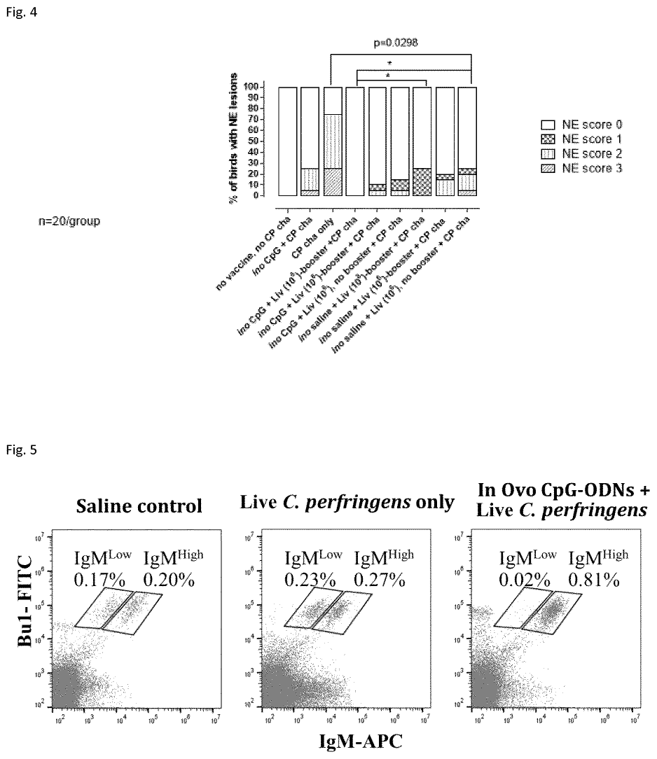

TABLE 3
Study design of animal experiment (results shown in FIGS. 3 and 4)

| | | | | | Challenge |
|---|---|---|---|---|---|
| | | | IPL delivery of live | Booster dose | (day 20-21- |
| Group | Number | in ovo @ day 18- | CP at the day of | at 10day of | 22) (CP in |
| # | of birds | incubation | hatch | age | feed) |
| 1 | 20 | Saline | − | − | − |
| 2 | 20 | CpG-ODN@50 | − | − | + |
| | | μg/egg | | | |
| 3 | 20 | Saline (100 μl)/egg | − | − | + |
| 4 | 20 | CpG-ODN@50 | Live CP (1 × 10⁸ cfu) | 1 × 10⁸ cfu | + |
| | | μg/egg | | inactivated | |
| | | | | CP with 10 μg | |
| | | | | of CpG-ODN | |
| 5 | 20 | CpG-ODN@50 | Live CP (1 × 10⁶ cfu) | 1 × 10⁸ cfu | + |
| | | μg/egg | | inactivated | |
| | | | | CP with 10 μg | |
| | | | | of CpG-ODN | |
| 6 | 20 | CpG-ODN@50 | Live CP (1 × 10⁶ cfu) | − | + |
| | | μg/egg | | | |
| 7 | 20 | Saline (100 μl)/egg | Live CP (1 × 10⁸ cfu) | 1 × 10⁸ cfu | + |
| | | | | inactivated | |
| | | | | CP with 10 μg | |
| | | | | of CpG-ODN | |
| 8 | 20 | Saline (100 | Live CP (1 × 10⁶ cfu) | 1 × 10⁸ cfu | + |
| | | μg/egg | | inactivated | |
| | | | | CP with 10 μg | |
| | | | | of CpG-ODN | |
| 9 | 20 | Saline (100 μl)/egg | Live CP (1 × 10⁶ cfu) | − | + |

The following is a description of the groups in the animal experiment results which are shown in FIG. 4. The objective of this experiment was to titrate dose of OP in the IPL vaccine against NE.
Group 1: This group is a control group with no intervention received.
Group 2: This group received in ovo delivery of CpG-ODN and CP challenge (CpG-ODN control)
Group 3: Positive control for necrotic enteritis (NE). Broilers were challenged with CP. This group is used as a comparison with all other groups to study challenge protection against CP.
Group 4: Eggs received 50 μg CpG-ODN by the in ovo route at day 18 of incubation, to modulate the immune system before vaccination at the time of hatch. At hatch, birds were vaccinated with live CP (1×10⁸ cfu) with no adjuvant by the IPL route and received an inactivated CP (1×10⁸ cfu) booster with 10 μg CpG-ODN as an adjuvant by the SC route at day 10 of age. Hence, this group demonstrated effect of 1×10⁸ cfu of live CP by the IPL route and boost.
Group 5: Eggs received 50 μg CpG-ODN by the in ovo route at day 18 of incubation to modulate the immune system before vaccination at the time of hatch. At hatch, birds were vaccinated with live CP (1×10⁶ cfu) with no adjuvant by the IPL route and received an inactivated CP (1×10⁸ cfu) booster with 10 μg CpG-ODN as an adjuvant by the SC route at day 10 of age. Hence, this group demonstrated effect of 1×10⁶ cfu of CP by the IPL route.
Group 6: Eggs received 50 μg CpG-ODN by the in ovo route at day 18 of incubation to modulate the immune system before vaccination at the time of hatch. At hatch, birds were vaccinated with live CP (1×10⁸ cfu) with no adjuvant by the IPL route and no CP booster vaccine. Hence, this group demonstrated induction of protective immunity by 1×10⁶ cfu of CP by the IPL route.
Group 7: Eggs received saline by the in ovo route at day 18 of incubation. At hatch, birds were vaccinated with live CP (1×10⁸ cfu) and boostered with 10 μg CpG-ODN as an adjuvant by the SC route at day 10 of age. Hence, this group demonstrated induction of protective immunity by 1×10⁸ cfu of CP by the IPL route with no immunomodulation with CpG-ODN.
Group 8: Eggs received saline by the in ovo route at day 18 of incubation. At hatch, birds were vaccinated with live CP (1×10⁶ cfu) and boostered with 10 μg CpG-ODN as an adjuvant by the SC route at day 10 of age. Hence, this group demonstrated induction of protective immunity by 1×10⁶ cfu of CP by the IPL route and boosting with 1×10⁸ cfu of CP.
Group 9: Eggs received saline by the in ovo route at day 18 of incubation. At hatch, birds were vaccinated with live CP (1×10⁶ cfu) and no booster vaccine. Hence, this group demonstrated induction of protective immunity by 1×10⁶ cfu of CP by the IPL route.

Conclusions: Protective immunity against NE was provided with live CP vaccines delivered by the IPL (1×10⁶ cfu and 1×10⁸ cfu) route with or without CpG-ODN delivery by the in ovo route. Protection against NE was provided by with or without CP booster vaccine following IPL vaccine.

### Flow Cytometry

Samples of blood (peripheral blood mononuclear cells (IPBMCs) and spleens were collected from respective groups at day 9 (before CP boost) and 24 days of age (trial end) for flow cytometry. Spleens (n=6/group) were collected aseptically and were kept on ice. Cell preparation and FACS staining was done as previously described with some modifications. Briefly, tissues were excised, and cell suspensions were prepared by crushing organs using a steel mesh and rubber plunger of a syringe head. Cell suspensions were washed thrice with FACS wash buffer (PBS containing 2% fetal bovine serum and 0.1% sodium azide). 1×RBC lysis buffer was added to cell pellet for 5 minutes or more depending on the lysis of RBCs on ice. Washing steps were repeated to remove lysis buffer and cells were divided for staining. Cells were then stained with antibodies (CD4+ PE-1 μl/sample, CD8+ FITC-1.5 μl/sample, IgM-APC-2 μl/sample, Bu1-488-1.5 μl/sample, monocyte-macrophage PE-1 μl/sample) at 4° C. for 30 min in dark on ice. After cells were washed and stained with secondary antibody (PerCP/cy 5.5 streptavidin-1 μl/sample) at 4° C. for 30 min in dark on ice. Following incubation cells were washed thrice before flow cytometry. Three ml of blood from each bird (n=6/group) were collected in heparinized tubes and kept at room temperature (18-26° C.) until processing. Afterwards, blood and PBS were mixed by the pipet at the ratio of 1:1 then carefully layered on 3 mL of histopaque and centrifuged for 45 minutes at 400G at 25° C. The PBMC layer was collected and washing steps were repeated. The rest of the steps were same as for the processing of spleens.

### Results:

### Mortality and NE Lesions:

The results shown in FIGS. 3 and 4 are discussed herein and the groups are described in Table 3. There was no mortality observed in group 1 (saline) and all vaccinated groups throughout the experiment while 15% and 10% mortality were observed in group 2 (in ovo CpG-ODN+CP challenge) and group 3 (CP challenge only) after 2 days post CP challenge. No NE lesions were observed in group 1 and 4 while 25%, 68%, 10%, 15%, 20%, 15% and 20% birds had NE lesions in groups 2, 4, 5, 6, 7, 8 and 9, respectively. A significant reduction in NE lesions was observed in vaccinated groups (p<0.001). The serum IgY response against CP vaccine was significantly higher in all vaccinated groups compared to the non-CP-vaccinated groups while mucosal IgA was significantly higher in group 4 (in ovo CpG-ODN+live CP IPL (1×10⁸)) compared to the other vaccinated groups.

Live CP (1×10⁸ cfu/bird) vaccination at hatch by the IPL route following in ovo CpG-ODN and inactivated CP boost (1×10⁸ cfu/bird with 10 μg of CpGODN) at 10 days of age produced highest amount of IgA and elevated IgG response. This group had no NE lesions (0%). Groups that received live CP vaccine (1×10⁸ cfu/bird) and CP boost (1×10⁸ cfu/bird with 10 μg CpG-ODN) with no in ovo CpG-ODN and the group that received live CP vaccine (1×10⁶ cfu) and CP boost (1×10⁶ cfu/bird with 10 μg CpG-ODN) with no in ovo CpG-ODN had elevated IgG.

Flow cytometry results showed a significant (p=0.04) increase in the IgM^{high} B cells in PBMC's and spleens in vaccinated groups administered only the live C. perfringens vaccine and in vaccinated groups administered in ovo CpG-ODNs and the live C. perfringens vaccine compared to the unvaccinated groups (FIG. 5).

### Conclusions:

In ovo CpG-ODN administration followed by 1×10⁸ cfu of live CP by the IPL delivery and CP booster with 10 μg of CpG-ODN as an adjuvant at 10 days of age provided a better protection against NE than the in ovo CpG-ODN followed by 1×10⁶ live CP by the IPL delivery and CP booster with 10 μg of CpG-ODN as an adjuvant at 10 days of age.

In ovo CpG-ODN administration followed by 1×10⁶ cfu of live CP by the IPL delivery without CP booster provided a significant protection against NE.

1×10⁸ or 1×10⁶ cfu of live CP by the IPL delivery and CP booster with 10 μg of CpG-ODN as an adjuvant at 10 days of age provided a protection against NE with no in ovo CpG-ODN.

1×10⁶ cfu of live CP by the IPL delivery without CP booster and with no in ovo CpG-ODN provided a significant protection against NE.

### Example 3

### Effects of IPL Delivery of Novel Live CP Vaccine Against Homologous and Heterologous CP Isolates Producing NE in Broiler Chickens

The objective of this experiment was to investigate immunoprotective effects of IPL delivery of live CP vaccine following in ovo CpG-ODN and boosting with inactivated CP with 10 μg of CpG-ODN by the subcutaneous (SC) route at 10 days of age against homologous and heterologous strains of CP associated with NE, to investigate immunoprotective effects of IPL delivery of live CP vaccine with no in ovo CpG-ODN or subcutaneous (SC) booster against homologous and heterologous strains of CP associated with NE, and to investigate immunoprotective effects of SC delivery of inactivated CP vaccine at hatch and boosting at 10 days of age following in ovo CpG-ODN against NE. CpG-ODN 2007 (SEQ ID NO: 1) was used in all formulations including CpG-ODNs in this example.

### Material and Methods

### Experimental Design

This work was approved by the AREB, University of Saskatchewan and adhered to the guidelines of the Canadian Council on Animal Care. Eggs (n=756) were incubated at the ACU, WCVM, University of Saskatchewan. Incubating eggs were candled at day 11 and 18 of incubation to confirm viability. At day 18 of incubation, fertile eggs were randomly assigned to ten groups (40 eggs per group). Incubating eggs were assigned as: 1) no vaccine and no CP challenge; 2) CP challenge only; 3) in ovo CpG-ODN+inactivated CP by the SC route at hatch and 10 days of age+CP challenge; 4) in ovo saline+inactivated CP by the SC route at hatch and 10 days of age+CP challenge; 5) in ovo CpG-ODN+IPL live CP (3 toxin strain-heterologous)+inactivated CP boost with 10 μg of CpG-ODN at 10 days of age+CP heterologous challenge; 6) in ovo saline+IPL live CP (3 toxin-heterologous)+inactivated CP boost with 10 μg of CpG-ODN at 10 days of age+CP heterologous challenge; 7) in ovo CpG-ODN+IPL live CP (4 toxin)+inactivated CP boost with 10 μg of CpG-ODN at 10 days of age+CP homologues challenge; 8) in ovo saline+IPL live CP (4 toxin)+inactivated CP boost with 10 μg of CpG-ODN at 10 days of age+CP homologues challenge; 9) in ovo CpG-ODN+IPL live CP (4 toxin-heterologous)+inactivated CP boost with 10 μg of CpG-ODN at 10 days of age+CP heterologous challenge; 10) in ovo saline+live CP (4 toxin-heterologous)+inactivated CP boost with 10 μg of CpG-ODN at 10 days of age+CP heterologous CP challenge. All CP live or inactivated vaccines contain 1×10⁸ cfu per bird. Additional groups of birds were vaccinated but not challenge with CP to take serum or intestinal scrapings to measure IgG or IgA (n=10/group). Groups that received CpG-ODN by the in ovo route delivered with 50 μg/egg CpG-ODN in 100 μl into the amniotic cavity using 23G 1 inch needle. Similarly, other groups received 100 μl of saline. Melted wax was used to seal the in ovo injection site following CpG-ODN administration. At day of hatch chicks in groups 5, 6, 7, 8, 9 and 10 were nebulized with lyophilized live CP (1×10⁸/bird) and groups 3 and 4 were injected with inactivated CP (1×10⁸/bird) by the SC route. The lung tissues (n=3) were collected at 24, 48, and 72 h post nebulization from each nebulized group. All the groups except group 1 and 2 were boosted with inactivated CP (1×10⁸/bird) formulated with 10 μg of COpG-ODN as an adjuvant by the SC route. All the groups except group 1 were challenged in feed with OP (1:1 ratio of 1×10⁹ cfu/g feed) at 20, 21 and 22 days of age. For histopathology, sections of intestines were collected. To measure IgG or IgA, blood or intestinal mucosal scrapings were collected at day 23 and 30 of age.

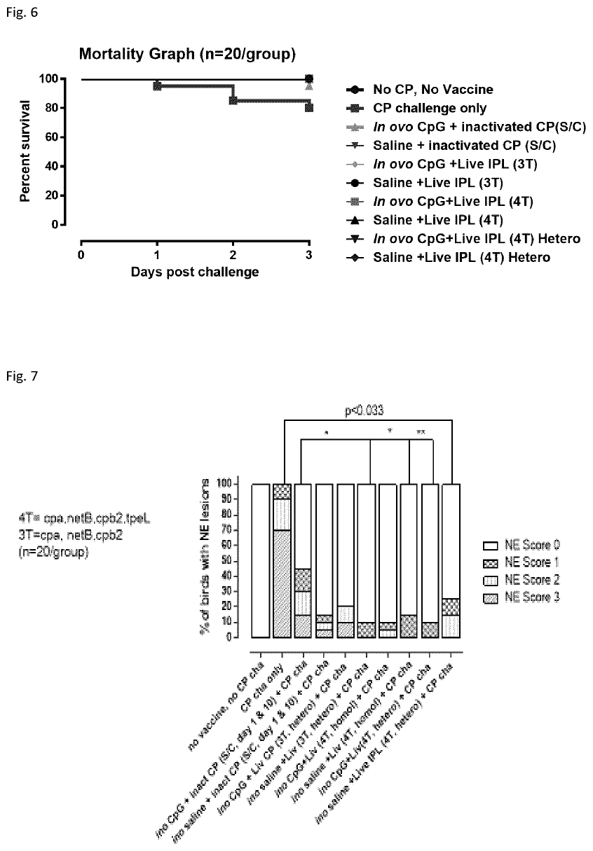

TABLE 4
Study design of animal experiment (results from FIGS. 6 and 7).

| | | | | | | Challenge |
|---|---|---|---|---|---|---|
| | | in ovo at | SC vaccination | | | (CP strain |
| Group | Number | day 18- | at the day of | Intrapulmonary at the | Booster dose | with 4 |
| # | of birds | incubation | hatch | day of hatch | at 10 d of age | toxin genes) |
| 1 | 20 | Saline | − | − | − | − |
| 2 | 20 | Saline | − | − | − | + |
| 3 | 20 | CpG@50 | CpG @ 10 μg + | − | Inactivated | + |
| | | μg/egg | inactivated CP | | CP (1 × 10⁸ | |
| | | | (10⁸ cfu) 4 toxin | | cfu) with 10 μg | |
| | | | strain | | CpG-ODN | |
| 4 | 20 | Saline | CpG @ 10 μg + | − | Inactivated | + |
| | | | inactivated CP | | CP (1 × 10⁸ | |
| | | | (10⁸ cfu) 4 toxin | | cfu) with 10 μg | |
| | | | strain | | CpG-ODN | |
| 5 | 20 | CpG@50 | − | Live CP (1 × 10⁸ cfu) | Inactivated | + |
| | | μg/egg | | (CP strain with 3 | CP (1 × 10⁸ | |
| | | | | toxin genes-cpa, | cfu) with 10 μg | |
| | | | | netB and cpb2)- | CpG-ODN | |
| | | | | heterologous | | |
| 6 | 20 | Saline | − | Live CP (1 × 10⁸ cfu) | Inactivated | + |
| | | | | (CP strain with 3 | CP (1 × 10⁸ | |
| | | | | toxin genes)- | cfu) with 10 μg | |
| | | | | heterologous | CpG-ODN | |
| 7 | 20 | CpG@50 | − | Live CP (1 × 10⁸ cfu) | − | + |
| | | μg/egg | | (CP strain with 4 | | |
| | | | | toxin genes)- | | |
| | | | | homologues | | |
| 8 | 20 | Saline | − | Live CP (1 × 10⁸ cfu) | − | + |
| | | | | (CP strain with 4 | | |
| | | | | toxin genes)- | | |
| | | | | homologues | | |
| 9 | 20 | CpG@50 | − | Live CP (1 × 10⁸ cfu)) | Inactivated | + |
| | | μg/egg | | CP strain with 4 toxin | CP (1 × 10⁸ | |
| | | | | genes)- | cfu) with 10 μg | |
| | | | | heterologous | CpG-ODN | |
| 10 | 20 | Saline | − | Live CP (1 × 10⁸ cfu)) | Inactivated | + |
| | | | | CP strain with 4 toxin | CP (1 × 10⁸ | |
| | | | | genes)- | cfu) with 10 μg | |
| | | | | heterologous | CpG-ODN | |

The following is a description of the groups involved in the experiment results for which are shown in FIG. 7. The objectives of this experiment were to demonstrate induction of protective immunity by the IPL route against homologous and heterologous protection and immunomodulatory activity CpG-ODN by in ovo delivery.
Group 1: This group is a control group with no intervention (see bar 1 in the FIG. 7).
Group 2: This group is a positive control for NE. Broilers were challenged with CP. This group is used a comparison with all other groups to study challenge protection against CP (see bar 2 in the FIG. 7).

Groups 3 and 4 demonstrate immunoprotection against CP by vaccinating birds by the subcutaneous (SC) route at hatch and boosting at 10 days of age by the SC route. These groups were included in this study as a comparator. There is no practical value here since birds cannot be vaccinated by the SC route at the hatchery and in the field at 10 days of age. The SC route was used as a comparison against the live CP vaccine administered via the IPL route described herein.
Group 3: Eggs received 50 μg CpG-ODN by the in ovo route at day 18 of incubation to modulate the immune system before vaccination at the time of hatch. At hatch, birds were vaccinated with inactivated CP with 10 μg CpG-ODN as an adjuvant by the subcutaneous (SC) route and received an inactivated CP booster with 10 μg CpG-ODN as an adjuvant by the SC route at day 10 of age. The vaccine and challenge CP strain contain 4 toxin genes (alpha, beta, netB, TPL) Hence, this group demonstrated homologous challenge protection following immunomodulation with CpG-ODN (see bar 3 in the FIG. 7).
Group 4: Eggs received saline by the in ovo route at day 18 of incubation. This is the control group for Group 3 to demonstrate effect of CpG-ODN by the in ovo route. At hatch, birds were vaccinated with inactivated CP by the SC route with 10 μg CpG-ODN as an adjuvant and received an inactivated CP booster with 10 μg CpG-ODN as an adjuvant by the SC route at day 10 of age. The vaccine and challenge CP strain contain 4 toxin genes (alpha, beta, netB, TPL) Hence, this group demonstrated homologous challenge protection with no immunomodulation CpG-ODN (see bar 4 in the FIG. 7).

Conclusion: Both groups 3 and 4 were protected against CP challenge at a significant level due to combination of COpG-ODN as an adjuvant in the CP vaccine and immunomodulation by in ovo delivery of CpG-ODN or CpG-ODN as an adjuvant in the CP vaccine.

Groups 5 and 6 were included in this study to demonstrate use of the IPL vaccine delivery technique with live CP at hatch following immunomodulation with CpG-ODN by the in ovo route and boosting with inactivated CP at 10 days of age with CP vaccine formulated with CpG-ODN as an adjuvant. Broilers cannot practically be boosted at 10 days of age in the field but groups 5 and 6 were control for groups for groups 7 and 8 (with no inactivated CP vaccination at 10 days of age).
Group 5: Eggs received 50 μg CpG-ODN by the in ovo route at day 18 of incubation to modulate the immune system before vaccination at the time of hatch. At hatch, birds were vaccinated with live CP with no adjuvant by the IPL route and received an inactivated CP booster with 10 μg CpG-ODN as an adjuvant by the SC route at day 10 of age. The IPL vaccine and the boost had a CP strain containing three toxin genes (alpha, beta, netB) and the CP challenge was performed with a CP strain containing 4 toxin genes (alpha, beta, netB, TPL). Hence, this group demonstrated heterologous challenge protection following immunomodulation with CpG-ODN by the in ovo route (see bar 5 in the FIG. 7).
Group 6: Eggs received saline by the in ovo route at day 18 of incubation. This is the control group for Group 5 to demonstrate the effect of CpG-ODN by the in ovo route. At hatch, birds were vaccinated with live CP with no adjuvant and received an inactivated CP booster with 10 μg CpG-ODN as an adjuvant by the SC route at day 10 of age. The IPL vaccine and the booster had a CP strain containing three toxin genes (alpha, beta, netB) and the CP challenge was performed with a CP strain containing 4 toxin genes (alpha, beta, netB, TPL). Hence, this group demonstrated heterologous challenge protection with no immunomodulation with CpG-ODN by the in ovo route (see bar 6 in the FIG. 7).

Conclusions: Both groups 5 and 6 were protected against CP at a significant level due to combination of live CP delivery by the IPL route and immunomodulation by in ovo delivery of CpG-ODN or by live CP delivery by the IPL route alone with no CpG-ODN mediated immunomodulation. Furthermore, these groups show IPL delivery can induce heterologous protection.

Groups 7 and 8 were included in this study to demonstrate the IPL vaccine delivery technique with live CP at hatch following immunomodulation with CpG-ODN by the in ovo route and no boosting. This novel IPL technique is practical in the poultry industry.
Group 7: Eggs received 50 μg CpG-ODN by the in ovo route at day 18 of incubation to modulate the immune system before vaccination at the time of hatch. At hatch, birds were vaccinated with live CP with no adjuvant by the IPL route. The IPL vaccine and challenge CP strain contained 4 toxin genes (alpha, beta, netB, TPL). Hence, this group demonstrated homologous challenge protection following immunomodulation with CpG-ODN by the in ovo route and induction of protective immunity following IPL vaccine (see bar 5 in the FIG. 7).
Group 8: Eggs received saline by the in ovo route at day 18 of incubation. This is the control group for Group 7 to demonstrate effect of CpG-ODN by the in ovo route. At hatch, birds were vaccinated with live CP with no adjuvant. The IPL vaccine and challenge CP strain contained 4 toxin genes (alpha, beta, netB, TPL). Hence, this group demonstrated homologous challenge protection with no immunomodulation with CpG-ODN by the in ovo route but induced protective immunity with IPL vaccine (see bar 6 in the FIG. 7).

Conclusion: Both groups 7 and 8 were protected against CP at a significant level. In group 7 this protection was due to combination of live CP delivery by the IPL route following CpG-ODN by the in ovo delivery. In group 8, where live CP was delivered by the IPL route with no in ovo CpG-ODN by delivery, this protection was due to induction of protective immunity by the IPL vaccine. Furthermore, this experiment shows a single IPL vaccine can protect birds with no booster for the duration of a broiler's life.

Similarly to groups 5 and 6, groups 9 and 10 were included in this study to study heterologous protection against CP by live CP vaccination by the IPL route following immunomodulation with CpG-ODN by the in ovo route. Boosting with inactivated CP was performed at 10 days of age with CP vaccine formulated with CpG-ODN as an adjuvant as in groups 5 and 6.
Group 9: Eggs received 50 μg CpG-ODN by the in ovo route at day 18 of incubation to modulate the immune system before vaccination at the time of hatch. At hatch, birds were vaccinated with live CP with no adjuvant by the IPL route and received an inactivated CP booster with 10 μg CpG-ODN as an adjuvant by the SC route at day 10 of age. The IPL vaccine had 4 toxin genes (alpha, beta, netB, TPL) but this is a different CP strain compared to the CP challenge strain. Hence, this group demonstrated heterologous challenge protection following immunomodulation with CpG-ODN by the in ovo route following induction of protective immunity by the IPL vaccine (see bar 9 in the FIG. 7).
Group 10: Eggs received saline by the in ovo route at day 18 of incubation. This is the control group for Group 9 to demonstrate the effect of CpG-ODN by the in ovo route. At hatch, birds were vaccinated with live CP with no adjuvant and received an inactivated CP booster with 10 μg CpG-ODN as an adjuvant by the SC route at day 10 of age. The IPL vaccine and challenge CP strain contain 4 toxin genes (alpha, beta, netB, TPL) but this is a different CP strain as compared to the CP challenge strain. Hence, this group demonstrated heterologous challenge protection with no immunomodulation with CpG-ODN by the in ovo route but induction of protective immunity by the IPL vaccine (see bar 10 in the FIG. 7).

Conclusions: Both groups 9 and 10 were protected against CP at a significant level due to combination of live CP delivery by the IPL route and immunomodulation by in ovo delivery of CpG-ODN or by live CP delivery by the IPL route alone with no CpG-ODN mediated immunostimulation. Furthermore, these groups show IPL vaccine delivery can induce immunity against heterologous protection.

### Flow Cytometry

The samples of blood (PBMC) were collected from group 1, 3, 4, 6 and 9 at day 9 (before CP booster) and 30 days of age (trial end). Three ml of blood per bird (n=6/group) were collected in heparinized tubes and kept at room temperature (18-26° C.) until processing. Then blood and PBS were mixed using a pipet in the ratio of 1:1 and carefully layered on 3 ml of histopaque and centrifuged for 45 minutes at 400G at 25° C. The PBMC layer was collected and washing steps were repeated. Cells were then stained with antibodies (CD4+ PE-1 μl/sample, CD8+ FITC-1.5 μl/sample, IgM-APC-2 μl/sample, Bu1-488-1.5 μl/sample, Monocyte-Macrophage PE-1 μl/sample) and incubated at 4° C. for 30 min in dark. After cells were washed and stained with secondary antibody (PerCP/cy 5.5 Streptavidin-1 μl/sample) and incubate at 4° C. for 30 min in dark. Following incubation, cells were washed thrice before flow cytometry analysis.

### Results

### Mortality and NE Lesions:

The results shown in FIGS. 6 and 7 are discussed herein and the groups are described in Table 4. There was no mortality observed in group 1 and vaccinated groups throughout the experiment except group 3 (in ovo COpG-ODN+Inactivated CP by the SC route) where 5% mortality was observed 2 days following CP challenge. Twenty percent (20%) mortality was observed in the group 2 (CP challenge only).

Flow cytometry demonstrated a significant (p=0.04) increase in the IgM^{high} B cells in vaccinated groups both before and after booster vaccination compared to the unvaccinated groups.

### Conclusions

In these experiments, it has been shown that IPL vaccine delivery can “induce” protective immunity (i.e., groups 5-6 and 9-10) against heterologous CP strains and this induction of immunity can also be boosted by a booster vaccination. It has also been shown that IPL vaccine delivery can induce protective immunity and protect birds against CP with no booster against homologous CP strains (groups 7-8). Based on the data, a booster provides a high level of antibodies against CP, particularly in the blood which is not important in NE since it is a disease in the intestine. In order to protect birds against NE, mucosal immunity (e.g. in the intestine) is relevant. CP delivery by the IPL route in combination with in ovo CpG-ODN or IPL delivery alone induced mucosal immunity much better than the current gold standard (group 3-4; provided systemic immunity-in the blood).

IPL delivery of live CP vaccine following in ovo CpG-ODN (50 μg/bird) and boosting with inactivated CP antigens (10 μg of CpG-ODN as an adjuvant) by the SC route at 10 days or CP vaccine with or without in ovo CpG-ODN with no booster (group 7 and 8) significantly protected broiler chickens against NE caused by homologous or heterologous strains of CP (FIGS. 5-6 and 7-8 and Table 4).

IPL delivery of live CP vaccine with no in ovo CpG-ODN but boosting with inactivated CP antigens with CpG-ODN as an adjuvant significantly protected broiler chickens against NE caused by homologous or heterologous strains of CP (FIGS. 5-6 and 9-10 and Table 4). A single vaccine with live CP at hatch can protect broilers against homologous and heterologous strains of CP (with or without in ovo CpG-ODN) and no booster needed.

SC delivery of inactivated CP vaccine (formulated with 10 μg of CpG-ODN as an adjuvant) at hatch and boosting (formulated with 10 μg of CpG-ODN as an adjuvant) at 10 days of age and following in ovo CpG-ODN or saline protected broiler chickens against NE at a significant level (FIG. 2). However, SC vaccination at the day of hatch or boosting at 10 days of age is not an industry feasible technique.

IPL delivery of live CP vaccine with or without in ovo CpG-ODN or with no CP booster vaccine is a technique that is feasible to perform in the industry and protected broiler chickens against CP (FIGS. 3 and 4).

### Example 4

The objective of this experiment was to compare three concentrations of sucrose and Mannitol solutions (1%, 10%, and 25%) as cryoprotectants for lyophilization of Clostridium perfringens and Escherichia coli using colony counts before and after lyophilization.

### Methodology

### Preparation of Different Concentrations Sucrose and Mannitol Solutions Used as Cryoprotectants

1%, 10%, and 25% solutions of each sugar type (100 ml each) were prepared following the protocol.

For preparation of 1% sugar solutions, add 1 g of sugar to a 125 ml sterile bottle and fill with distilled water up to 100 ml mark. Mix and autoclave at 121° C. for 15 minutes.

For preparation of 10% sugar solutions, add 10 g of sugar to a 125 ml sterile bottle and fill with distilled water up to 100 ml mark. Mix and autoclave at 121° C. for 15 minutes.

For preparation of 25% sugar solutions, add 25 g of sugar to a 125 ml sterile bottle and fill with distilled water up to 100 ml mark. Mix and autoclave at 121° C. for 15 minutes.

### Bacterial Isolation

Streak CP21 and E. coli separately on Columbia 5% sheep blood agar plates. Incubate CP21 anaerobically at 37° C. for 24 hours. Incubate E. coli at 37° C. for 24 hours. (No anaerobic conditions). After 24 hrs. inoculate 1 colony of CP21 in 100 ml TGM and Incubate anaerobically at 37° C. for 15-16 hours. Inoculate 1 colony of E. coli in 100 ml Luria broth and incubate at 37° C. for 16-18 hours with shaking at 150 rpm. (No anaerobic conditions).

Harvesting of Bacteria (C. perfringens and E. coli) for Lyophilization

Transfer 20 ml of the live culture to 50 mL centrifuge tubes and centrifuge at 4000 rpm for 30 minutes. Discard the supernatant and wash the pellet with 20 ml PBS solution twice at 4000 rpm for 30 minutes. After washing, transfer the bacterial pellet required for 5 birds ie, 500 uL (100 uL/bird) into 500 μL of each 1%, 10%, and 25% sucrose in a 3 ml vaccine vials. Prepare a serial dilution from 50 microliters from each vial with different sugar concentrations and spread in 5% Columbia sheep blood agar in duplicates to obtain colony counts before lyophilization. Repeat the above procedure using the 1%, 10%, and 25% solutions of Mannitol. Plug the vials with cotton and store at −80° C. freezer until lyophilization.

### After Lyophilization,

Dissolve the vials corresponding to each sucrose concentration in 1 ml of sterile saline, count CFUs and measure the OD values. Repeat this step for Mannitol as well. Store other vials at 4° C. until the day of nebulization.

### Results

### CFU Counts Before and After Lyophilization

TABLE 4
Sucrose solutions

| | Before lyophilization | | | After lyophilization | | |
|---|---|---|---|---|---|---|
| | Sucrose | Sucrose | Sucrose | Sucrose | Sucrose | Sucrose |
| Bacterium | 1% | 10% | 25% | 1% | 10% | 25% |
| C. perfringens | 1.2 × 10⁸ | 1.0 × 10⁸ | 1.1 × 10⁸ | 3 × 10⁷ | 2.8 × 10⁷ | 3.2 × 10⁷ |
| E. coli | 3.2 × 10⁹ | 2.4 × 10⁹ | 1.0 × 10⁹ | 2 × 10⁹ | 2.3 × 10⁹ | 1 × 10⁹ |

TABLE 5
Mannitol solutions

| | Before lyophilization | | | After lyophilization | | |
|---|---|---|---|---|---|---|
| | Sucrose | Sucrose | Sucrose | Sucrose | Sucrose | Sucrose |
| Bacterium | 1% | 10% | 25% | 1% | 10% | 25% |
| C. perfringens | 1.2 × 10⁸ | 1.0 × 10⁸ | 1.1 × 10⁸ | 1.0 × 10⁷ | 1.0 × 10⁷ | 1.0 × 10⁷ |
| E. coli | 3.2 × 10⁹ | 2.4 × 10⁹ | 1.0 × 10⁹ | 1.7 × 10⁸ | 2 × 10⁹ | 1 × 10⁹ |

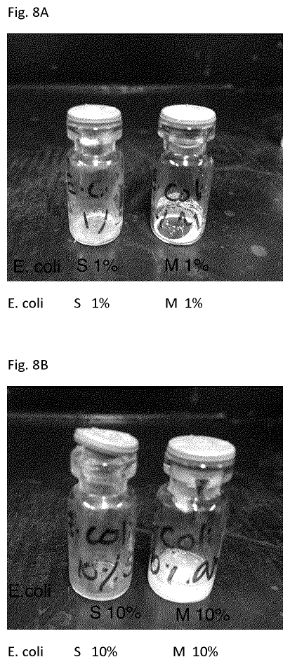

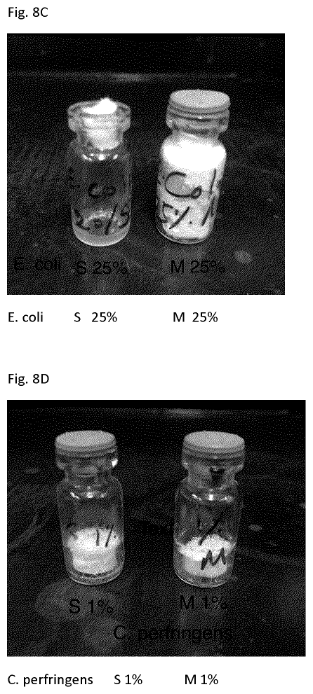

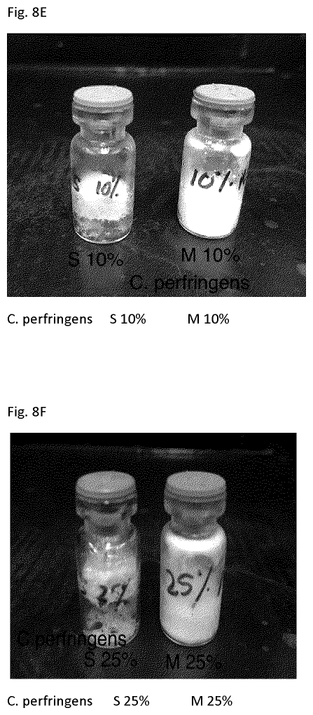

All solutions of sucrose and mannitol except 25% sucrose gave powdery products after lyophilization for both bacterium types. Sucrose 25% solution resulted in a gelatinous product after lyophilization (see FIGS. 8A-F).

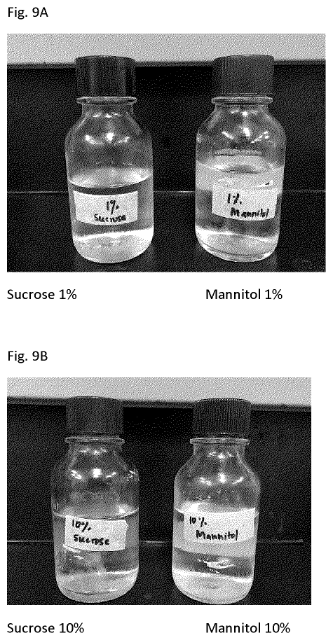

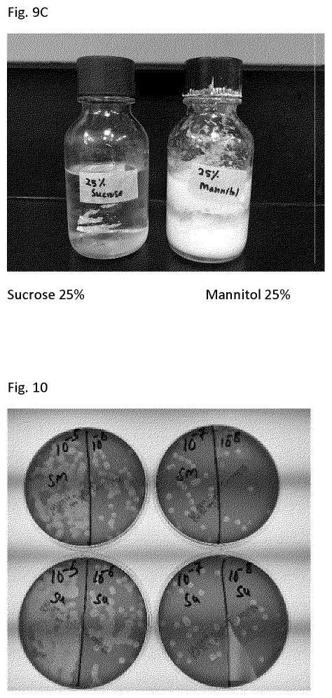

Further, all sugar solutions were colorless and liquid at the room temperature, while mannitol 25% solution was in white crystalline form (see FIGS. 9A-C).

TABLE 6
Optical Density values after lyophilization

| Sample | Absorbance at 600 |
|---|---|
| CP21 1% Sucrose | 3.163 |
| CP21 10% Sucrose | 3.076 |
| CP21 25% Sucrose | 3.078 |
| CP21 1% Mannitol | 3.394 |
| CP21 10% Mannitol | 3.173 |
| CP21 25% Mannitol | 1.936 |
| E.coli 1% Sucrose | 3.294 |
| E.coli 10% Sucrose | 3.238 |
| E.coli 25% Sucrose | 3.236 |
| E.coli 1% Mannitol | 3.189 |
| E.coli 10% Mannitol | 3.265 |
| E.coli 25% Mannitol | 3.346 |

### Example 5

C. perfringens (CP) Vaccine Preparation and Intrapulmonary Delivery (IPL)

### Inactivated CP Vaccine Preparation:

Single colony of C. perfringens was inoculated in 50 mL of fluid thioglycollate medium (Sigma-Aldrich) for 16 hours (hrs.) in 100 mL capacity Erlenmeyer flasks under anaerobic conditions (BD Gas Pak EZ chamber with 2 sachets of AnaeroGen 3.5 L, Thermo scientific) at 37° C. The following day, C. perfringens culture was inactivated with 0.04% formaldehyde incubated at 37° C. for overnight. To ensure inactivation of C. perfringens, 100 μL of overnight culture was plated on blood agar (Columbia Blood Agar with 5% defibrinated sheep blood, ThermoScientific). Inactivated culture solution was dispensed in 50 mL centrifuge tubes in 35 mL aliquots and centrifuged (Sorvall Legend RT, Mandel) at 4,000 rpm for 20 minutes. The supernatants were discarded, and bacterial pellets were washed twice with phosphate buffered saline (pH−7.4, 0.01M). Further, bacterial pellets were transferred to the 3 mL capacity sterile glass vials with and sucrose (10%) in the ratio of 1:1 and stored at −80° C. overnight till lyophilization (Labconco Freeze Dry System Freezone 2.5). After freeze drying, fine powder of C. perfringens was stored at 4° C. till use.

### Live CP Vaccine Preparation:

All the above steps were followed except formaldehyde inactivation. To confirm the viability of C. perfringens following lyophilization, lyophilized product serially diluted in phosphate buffered saline (PBS) and plated in blood agar for enumeration in duplicates. Live C. perfringens (1×10³ cfu/bird) vaccine was diluted in sterile 0.9% saline and aerosolized as microdroplets of 0.5 μm by using a Compressor Nebulizer (705-470) (AMG Medical Inc. Montreal, QC, Canada). For inactivated C. perfringens vaccine (1×10³ cfu/bird) synthetic CpG-ODN (TCGTCGTTGTCGTTTTGTCGTT, Class B, 2007) was used as an adjuvant (@10 μg) with a phosphorothioate backbone (Operon Biotechnologies, Inc. Huntsville, AL). Same procedure was followed for the live C. perfringens vaccine preparation for heterologous vaccine challenge. CpG-ODN 2007 (SEQ ID NO: 1) was used in all formulations including CpG-ODNs in this example.

### Lyophilization Media

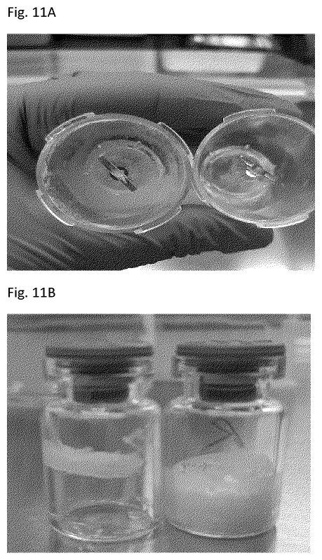

For aerosolization of C. perfringens two separate media were tested for the lyophilization (a) 10% skim milk and (b) 10% sucrose. To confirm the viability of C. perfringens following lyophilization, lyophilized product serially diluted in phosphate buffered saline (PBS) and plated on blood agar for enumeration in duplicates. Under our experimental conditions both media did not affect the viability of the C. perfringens and showed 7×10⁹ cfu/mL (FIG. 10). However, the miscibility and aerosolization was better with 10% sucrose compared to 10% skim milk. Upon aerosolization of C. perfringens lyophilized with 10% skim milk formed the froth under air pressure and 1 mL left at the bottom of the container in 15 minutes compared to the sucrose (FIG. 11A). In addition, pellet of C. perfringens with skim milk was bigger and viscous compared to the sucrose where it was more compact and smaller (FIG. 11B)

Confirmation of Vegetative Form of C. perfringens

Different methods were tested to inactivate the C. perfringens 1) heat inactivation at 65 C for one hour in the water bath and 2) 0.04% formaldehyde at 37° C. in incubator for overnight. Endospore staining (Schaeffer-Fulton stain technique) was performed to detect the spore formation by C. perfringens. There were no spores detected in both the methods.

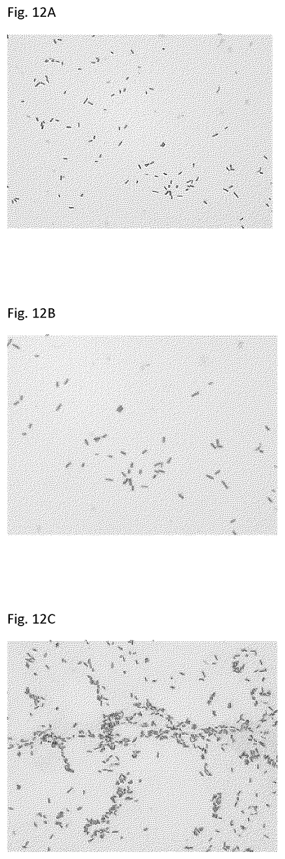

Endospore should stain green if present (none shown in FIGS. 12A-C) while vegetative bacterial cell should stain purple to pink (shown as grey in FIGS. 12A-C).

### Example 6

### Development of Innate Immune Memory by CpG-ODN Potentiate Immune Protection Against Necrotic Enteritis by IPL Delivery of CP Vaccine

### Experimental Design

The effect of administering CpG-ODN prior to intra-pulmonary (IPL) CP vaccine on immune cell metabolism, compared to the effect of CpG-ODN alone was investigated. CpG-ODN 2007 (SEQ ID NO: 1) was used in all formulations including CpG-ODNs in this example.

In ovo CpG-ODN was injected at 50 μg/egg at day 18 of incubation of embryonated eggs. At hatch fifteen birds were randomly divided into 3 groups (n=5/group) where Group 1 was administered in ovo CpG-ODN and live intrapulmonary (IPL) vaccine (C. perfringens 1×10⁸ cfu/mL); Group 2 was administered live IPL vaccine only; Group 3 was administered in ovo CpG-ODN only. At day of hatch, 200 μl of live C. perfringens vaccine was given via IPL route followed by a booster dose (inactivated CP with CpG-ODN 2007) at day 10 of the first vaccine dose. The 3 mL blood was collected from all the birds at 3, 14 and 21 days after the booster (dpb) dose using heparin coated blood collection tubes. The same birds were used for both time points to overcome individual bird variations as a longitudinal study. A mitochondrial-stress assay and glycolytic rate assay were performed at each time point.

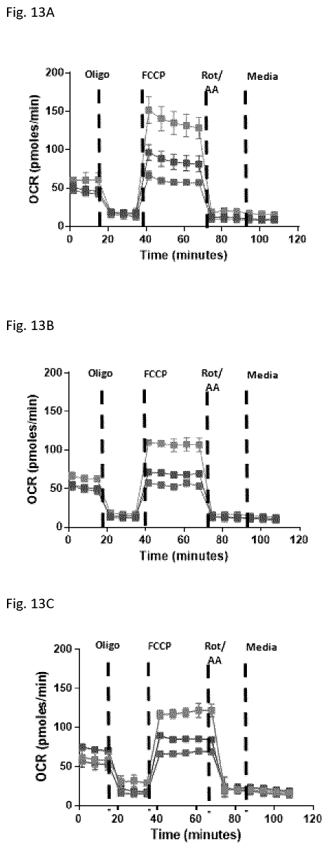

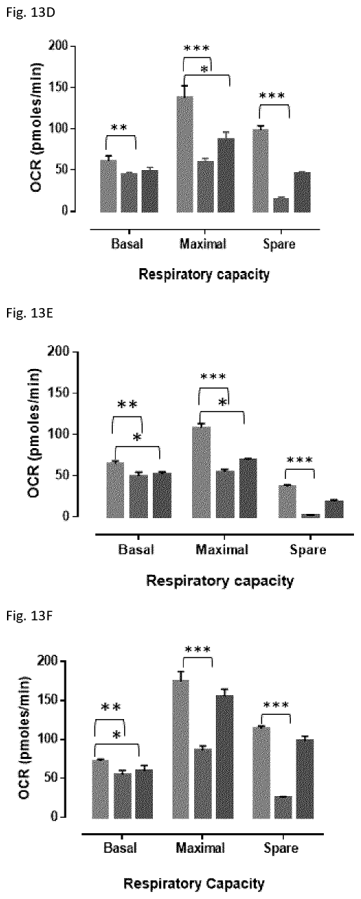

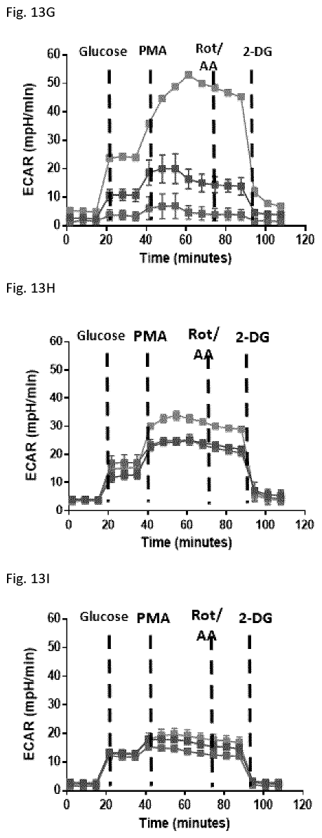

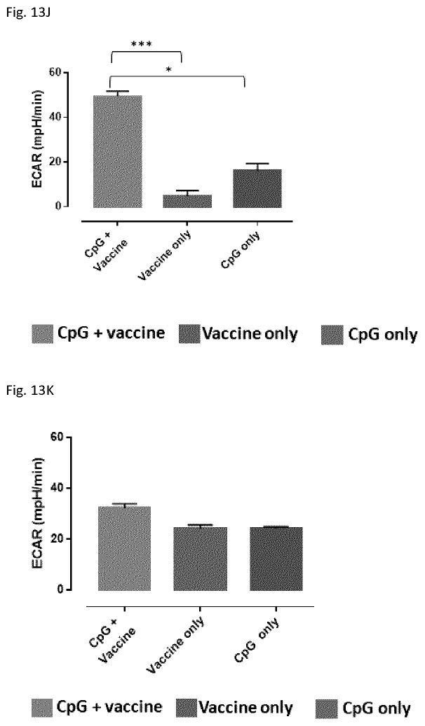

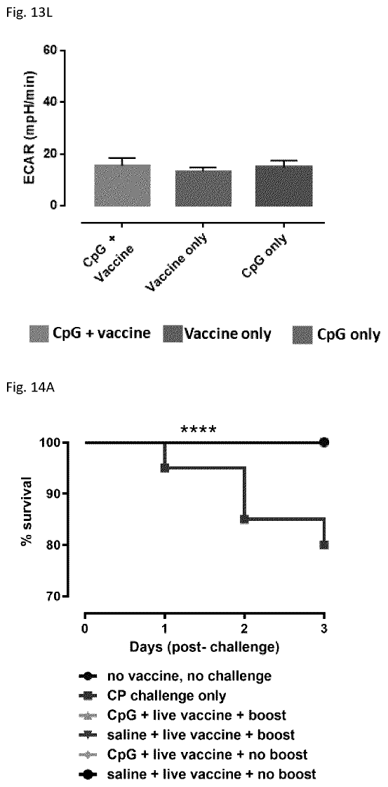

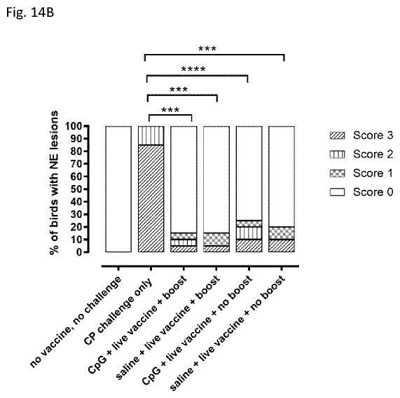

Outputs were collected at 3 time points. Kinetics of OXPHOS of mitochondria were shown as at (FIG. 13A) 3 dpb, (FIG. 13B) 14 dpb, and (FIG. 13C) 21 dpb. Basal, maximal, and spare respiratory capacity at (FIG. 13D) 3 dpb, (FIG. 13E) 14 dpb, and (FIG. 13F) 21 dpb were calculated from raw data. Changes of cellular glycolysis over time were shown as ECAR at (FIG. 13G) 3 dpb, (FIG. 13H) 14 dpb, and (FIG. 13I) 21 dpb. The compensation by glycolytic pathway (compensatory glycolysis) was shown in bar graphs at (FIG. 13J) 3 dpb, (FIG. 13K) 14 dpb, and (FIG. 13L) 21 dpb. Each data point represents the mean measurement obtained from 5 birds/group with two experiential replicates. Significant differences are indicated by * p<0.05.

### Results

Enhanced Effect on Mitochondrial OXPHOS and Glycolysis Pathways in Chicken PBMCs when Combined CpG-ODN with IPL CP Vaccine than the Effect of CpG-ODN Alone

This experiment was conducted to compare the mitochondrial OXPHOS and cellular glycolysis of PBMCs in birds administered a combination of CpG-ODN+live intrapulmonary (IPL) CP vaccine vs CpG-ODN only and vs vaccine-only groups. Blood samples were collected at 3 different time points; 3, 14, and 21 dpb. We observed a significantly increased OCR activity over the time up to 21 dpb in CpG-ODN+vaccine-administered birds than other groups (FIGS. 13A, B, and C). Compared to vaccine only group, basal (p=0.0029), maximal (p=0.0001), and spare respiratory capacity (p=0.0003) were significantly higher in CpG-ODN+vaccine group at 3 dpb. When compared with CpG-ODN only group values were significantly higher only in maximal respiration (p=0.0333) at 3 dpb (FIG. 13D). At 14 dpb, mitochondrial respiration was further elevated including basal (p=0.0035), maximal (p=0.0001), and spare respiration (p=0.0003) in the CpG-ODN+vaccine group than in the vaccine-only group. Compared to the CpG-ODN-only group, mitochondrial respiration was elevated only in basal (p=0.0283) and maximal respiration (p=0.0033) in the CpG-ODN+vaccine group (FIG. 1E). Mitochondrial activity was elevated and extended up to 21 dpb in CpG-ODN+vaccine birds with higher basal (p=0.0024), maximal (p=0.0001), and spare respiration (p=0.0003) than vaccine only group, however, only basal respiration was significantly higher than CpG-ODN only group (p=0.0386) (FIG. 13F). ECAR quantification showed a transient difference between groups (FIGS. 13G, H and I). Glycolysis of PBMC isolated from the CpG-ODN+vaccine group revealed significantly higher compensatory glycolysis than vaccine only (p=0.0001) and CpG-ODN only (p=0.0333) at 3 dpb (FIG. 13J). Despite this, differences were not remarkable between groups at later time points 14 and 21 dpb (FIGS. 13L and K).

### Conclusions

Birds that were administered with live CP vaccine by the IPL route followed by in ovo delivery of CpG-ODN were protected at a significant level (data not shown). This study was conducted to explore mechanisms associated with CpG-ODN mediated immunoprotection with live CP vaccine. Here we demonstrate that injecting CpG-ODN by the in ovo route and booster dose with live CP antigens was able to switch immune cells into memory cells (innate memory) by changing cellular energy dependence from glycolytic pathway to mitochondrial pathway.

### Example 7

Immunoprotective Effects of IPL Delivery of Live CP Vaccine Against Heterologous CP Associated with NE in Broiler Chickens without a Booster Vaccine

### Materials and Methods

The experiment was conducted to investigate immunoprotective effects of IPL delivery of live CP vaccine at day 1 of age with no CP booster vaccine against heterologous CP challenge associated with NE in broiler chickens. In order to compare effects of booster vaccination, two groups vaccinated with live CP by the IPL route were boosted with formalin inactivated whole cell CP antigens (1×10⁸ cfu/bird) formulated with 10 μg of CpG-ODN as an adjuvant at day 10 of age. Embryonated eggs were randomly assigned to groups: (1) no vaccination and no CP challenge; (2) CP challenge; (3) in ovo CpG-ODN+IPL live CP (isolate with 4 toxin genes, heterologous)+boost+CP challenge; (4) in ovo saline+IPL live CP (isolate with 4 toxin genes, heterologous)+boost+CP challenge; (5) in ovo CpG-ODN+IPL live CP (isolate with 4 toxin genes, heterologous)+no boost+CP challenge; (6) in ovo saline+IPL live CP (isolate with 4 toxin genes, heterologous)+no boost+CP challenge. Sections of intestines were collected for histopathology from any dead or euthanized birds at the termination of the experiment (n=20/group).

### RESULTS

Groups vaccinated with live CP by the IPL route following in ovo delivery of CpG-ODN or without CpG-DN and immunized with or without a booster CP vaccine had significantly low histopathological lesions of NE (p<0.0001) (FIG. 14B).

The group that received no CP vaccine and was not challenged with CP (group 1) had no gross or microscopic lesions of NE (0%). The group that received no CP vaccine but was challenged with CP (group 2) had 20% mortality. Groups vaccinated with live CP by the IPL route with or without CpG and with or without booster, and challenged with CP did not have any mortality (0%) (FIG. 14A).

As mentioned above, the group that received no CP vaccine and was not challenged with CP (group 1) had no gross or microscopic lesions of NE (0%). The group challenged with CP that did not receive a CP vaccine (group 2) had 100% of birds with macroscopic lesions (85% of birds with score 3) and microscopic lesions (15% of birds with score 2) following CP challenge. The group that received in ovo CpG-ODN and live CP by the IPL route with inactivated CP boost by the SC route (group 3) had 15% of birds with macroscopic (5% with score 3) and microscopic lesions (5% with score 1 and 5% with score 2) of NE following CP challenge. The group that received in ovo saline and live CP by the IPL route with inactivated CP boost by the SC route (group 4) had 15% of birds with macroscopic (5% with score 3) and microscopic lesions (10% with score 1) of NE following CP challenge. The group that received in ovo CpG-ODN and live CP by the IPL route with no CP boost (group 5) had 25% of birds with macroscopic (10% with score 3) and microscopic lesions (5% with score 1 and 10% with score 2) of NE following CP challenge. The group that received in ovo saline and live CP by the IPL route with no CP boost (group 6) had 20% of birds with macroscopic (10% with score 3) and microscopic lesions (10% with score 1) of NE following CP challenge. All the groups that received either in ovo CpG-ODN or saline and CP vaccine by the IPL route with or without CP booster vaccine had a statistically significant reduction in NE pathology (P<0.0001) (FIG. 14B).

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g., protein and/or nucleic acid) provided in the Tables or elsewhere, are incorporated by reference in its entirely.

The scope of the claims should not be limited by the preferred embodiments and examples but should be given the broadest interpretation consistent with the description as a whole.

### REFERENCES

1. Cooper, K. K., and J. G. Songer. Virulence of Clostridium perfringens in an experimental model of poultry necrotic enteritis. Veterinary microbiology 142: 323-328. 2010.
2. Helmboldt, C., and E. Bryant. The pathology of necrotic enteritis in domestic fowl. Avian Diseases: 775-780. 1971.
3. Keyburn, A. L., J. D. Boyce, P. Vaz, T. L. Bannam, M. E. Ford, D. Parker, A. Di Rubbo, J. I. Rood, and R. J. Moore. NetB, a new toxin that is associated with avian necrotic enteritis caused by Clostridium perfringens. PLoS pathogens 4: e26. 2008.
4. Kurukulsuriya, S., K. A. Ahmed, D. Ojkic, T. Gunawardana, A. Gupta, K. Goonewardene, R. Karunaratne, S. Popowich, P. Willson, and S. K. Tikoo. Circulating strains of variant infectious bursal disease virus may pose a challenge for antibiotic-free chicken farming in Canada. Research in Veterinary Science 108: 54-59. 2016.
5. Lanckriet, A., L. Timbermont, V. Eeckhaut, F. Haesebrouck, R. Ducatelle, and F. Van Immerseel. Variable protection after vaccination of broiler chickens against necrotic enteritis using supernatants of different Clostridium perfringens strains. Vaccine 28: 5920-5923. 2010.
6. Lee, K., H. Lillehoj, W. Jeong, H. Jeoung, and D. An. Avian necrotic enteritis: experimental models, host immunity, pathogenesis, risk factors, and vaccine development. Poultry Science 90: 1381-1390. 2011.
7. McDevitt, R., J. Brooker, T. Acamovic, and N. Sparks. Necrotic enteritis; a continuing challenge for the poultry industry. World's Poultry Science Journal 62: 221-247. 2006.
8. Mot, D., L. Timbermont, F. Haesebrouck, R. Ducatelle, and F. Van Immerseel. Progress and problems in vaccination against necrotic enteritis in broiler chickens. Avian pathology 43: 290-300. 2014.
9. Paiva, D., and A. McElroy. Necrotic enteritis: applications for the poultry industry. Journal of Applied Poultry Research 23: 557-566. 2014.
10. Ulmer-Franco, A., G. Cherian, N. Quezada, G. Fasenko, and L. McMullen. Hatching egg and newly hatched chick yolk sac total IgY content at 3 broiler breeder flock ages. Poultry science 91: 758-764. 2012.
11. Wade, B., and A. Keyburn. The true cost of necrotic enteritis. World Poult 31: 16-7. 2015.
12. Zachar, T., S. Popowich, B. Goodhope, T. Knezacek, D. Ojkic, P. Willson, K. A. Ahmed, and S. Gomis. A 5-year study of the incidence and economic impact of variant infectious bursal disease viruses on broiler production in Saskatchewan, Canada. Canadian Journal of Veterinary Research 80: 255-261. 2016.
13. Goonewardene K, Popowich S, Gebhardt S, Gunawardana T, Gupta A, Kurukulasuriya S, Karunarathna R, Liu M, Chow-Lockerbie B, Ayalew L, Ahmed K A, Kamali H, Tikoo S K, Foldvari M, Willson P, Boire J, Roberts K, Ambrose N, Simonson C and S Gomis. 2021. Aerosol delivery of synthetic DNA containing CpG motifs in broiler chicks at hatch under field conditions using a commercial-scale prototype nebulizer provided protection against lethal Escherichia coli septicemia. Poultry Science. https://doi.org/10.1016/j.psj/2020.12.031.
14. Corbanie E A, Vervaet C, van Eck J H, Remon J P, Landman W J. Vaccination of broiler chickens with dispersed dry powder vaccines as an alternative for liquid spray and aerosol vaccination. Vaccine. 2008 Aug. 18; 26(35):4469-76. doi: 10.1016/j.vaccine.2008.06.055. Epub 2008 Jul. 1. PMID: 18598731.

1. A method of eliciting an immune response in one or more subject(s) against Clostridium perfringens (C. perfringens) infection and/or necrotic enteritis, the method comprising administering to one or more subject(s) an amount of a composition comprising live C. perfringens, and optionally a pharmaceutically acceptable carrier or diluent.

2. A method of inducing protective immunity against or preventing Clostridium perfringens (C. perfringens) infection and/or necrotic enteritis, the method comprising administering to one or more subject(s) an amount of a composition comprising live C. perfringens, and optionally a pharmaceutically acceptable carrier or diluent.

3. The method of claim 1, wherein the composition is formulated for intrapulmonary administration.

4. The method of claim 2, wherein the composition is formulated for administration via nebulization.

5. The method of claim 1, wherein the composition is formulated as or to produce microdroplets.

6. The method of claim 5, wherein the microdroplets have a particle size of about 0.5 μm to about 5 μm.

7. The method of claim 1, wherein the concentration of live C. perfringens in the composition is about 0.5×10⁷ cfu to about 1.6×10¹⁰ cfu, between about 1×10⁷ cfu to about 1×10⁹ cfu, or is about 1×10⁹ cfu.

8. (canceled)

9. The method of claim 1, wherein the composition is administered to the one or more subject(s) for between about 15 to about 30 minutes, or about 30 minutes; and/or wherein the composition is administered in an environment having a temperature of about 25° C. to about 30° C., or about 28° C. to about 30° C.

10.-11. (canceled)

12. The method of claim 1, wherein the composition is administered in an environment wherein the humidity is less than about 70%, between about 40% and about 70%, or between about 40% and about 60%; and/or is administered prior to exposure to C. perfringens.

13. (canceled)

14. The method of claim 1, wherein one or more CpG oligodeoxynucleotide(s) is/are administered prior to administration of the composition, optionally CpG-ODN 2007.

15. The method of claim 14, wherein the one or more CpG-ODN(s) is administered in ovo, optionally at day 18 of incubation.

16. The method of claim 1, wherein the composition is resuspended from a lyophilized composition prior to administration.

17. The method of claim 16, wherein the composition further comprises sucrose or mannose.

18. (canceled)

19. The method of claim 16, wherein the composition lacks skim milk.

20. (canceled)

21. The method of claim 16, wherein the lyophilized composition is resuspended in water, optionally distilled water, phosphate buffered saline or saline prior to administration to the one or more subject(s).

22. (canceled)

23. The method of claim 1, wherein the composition is sterile.

24. The method of claim 1, wherein the one or more subject(s) is is/are poultry, wherein the one or more subject(s) is/are chicken, turkey, or duck, or wherein the one or more subject(s) is/are broiler chicken.

25.-27. (canceled)

28. The method of claim 24, wherein the composition is administered within about 24 hours to about 36 hours of hatch, or within about 24 hours of hatch.

29. The method of claim 24, wherein the administration to the one or more subject(s) is performed in a closed chamber.

30. The method of claim 1, wherein the amount of the live C. perfringens delivered or to be delivered per subject being administered the composition is about 1×10⁶ bacteria to about 1×10⁹ bacteria, or about 1×10⁷ bacteria to about 1×10⁸ bacteria, or about 1×10⁸.

31.-59. (canceled)
